SEQUENCE LISTING (2 sequences)

SEQ ID NO: 1 - protein, 535 aa, Homo sapiens
MGIVEPGCGDMLTGTEPMPGSDEGRAPGADPQHRYFYPEPGAQDADERRGGGSLGSPYPGGALVPAPPSRFLGAYAYPPRPQAAGFPGAGESFPPPADAEGYQPGEGYAAPDPRAGLYPGPREDYALPAGLEVSGKLRVALNNHLLWSKFNQHQTEMIITKQGRRMFPFLSFTVAGLEPTSHYRMFVDVVLVDQHHWRYQSGKWVQCGKAEGSMPGNRLYVHPDSPNTGAHWMRQEVSFGKLKLTNNKGASNNVTQMIVLQSLHKYQPRLHIVEVNDGEPEAACNASNTHIFTFQETQFIAVTAYQNAEITQLKIDNNPFAKGFRENFESMYTSVDTSIPSPPGPNCQFLGGDHYSPLLPNQYPVPSRFYPDLPGQAKDVVPQAYWLGAPRDHSYEAEFRAVSMKPAFLPSAPGPTMSYYRGQEVLAPGAGWPVAPQYPPKMGPASWFRPMRTLPMEPGPGGSEGRGPEDQGPPLVWTEIAPIRPESSDSGLGEGDSKRRRVSPYPSSGDSSSPAGAPSPFDKEAEGQFYNYFPN

SEQ ID NO: 2 - DNA, 1608 bp, Homo sapiens
atgggcatcgtggagccgggttgcggagacatgctgacgggcaccgagccgatgccggggagcgacgagggccgggcgcctggcgccgacccgcagcaccgctacttctacccggagccgggcgcgcaggacgcggacgagcgtcgcgggggcggcagcctggggtctccctacccggggggcgccttggtgcccgccccgccgagccgcttccttggagcctacgcctacccgccgcgaccccaggcggccggcttccccggcgcgggcgagtccttcccgccgcccgcggacgccgagggctaccagccgggcgagggctacgccgccccggacccgcgcgccgggctctacccggggccgcgtgaggactacgcgctacccgcgggactggaggtgtcggggaaactgagggtcgcgctcaacaaccacctgttgtggtccaagtttaatcagcaccagacagagatgatcatcaccaagcagggacggcggatgttcccattcctgtcatttactgtggccgggctggagcccaccagccactacaggatgtttgtggacgtggtcttggtggaccagcaccactggcggtaccagagcggcaagtgggtgcagtgtggaaaggccgagggcagcatgccaggaaaccgcctgtacgtccacccggactcccccaacacaggagcgcactggatgcgccaggaagtttcatttgggaaactaaagctcacaaacaacaagggggcgtccaacaatgtgacccagatgattgtgctccagtccctccataagtaccagccccggctgcatatcgttgaggtgaacgacggagagccagaggcagcctgcaacgcttccaacacgcatatctttactttccaagaaacccagttcattgccgtgactgcctaccagaatgccgagattactcagctgaaaattgataataacccctttgccaaaggattccgggagaactttgagtccatgtacacatctgttgacaccagcatcccctccccgcctggacccaactgtcaattccttgggggagatcactactctcctctcctacccaaccagtatcctgttcccagccgcttctaccccgaccttcctggccaggcgaaggatgtggttccccaggcttactggctgggggccccccgggaccacagctatgaggctgagtttcgagcagtcagcatgaagcctgcattcttgccctctgcccctgggcccaccatgtcctactaccgaggccaggaggtcctggcacctggagctggctggcctgtggcaccccagtaccctcccaagatgggcccggccagctggttccgccctatgcggactctgcccatggaacccggccctggaggctcagagggacggggaccagaggaccagggtccccccttggtgtggactgagattgcccccatccggccggaatccagtgattcaggactgggcgaaggagactctaagaggaggcgcgtgtccccctatccttccagtggtgacagctcctcccctgctggggccccttctccttttgataaggaagctgaaggacagttttataactattttcccaactga

(19) United States
(12) Patent Application Publication
(10) Pub. No.: US 2017/0326133 A1
(43) Pub. Date: Nov. 16, 2017

(54) METHODS OF TREATING MYELODYSPLASTIC SYNDROME WITH FARNESYLTRANSFERASE INHIBITORS

(71) Applicant: Kura Oncology, Inc., La Jolla, CA (US)

(72) Inventor: Antonio GUALBERTO, Acton, MA (US)

(21) Appl. No.: 15/590,513

(22) Filed: May 9, 2017

Related U.S. Application Data
(60) Provisional application No. 62/334,322, filed on May 10, 2016.

(51) Int. Cl. A61K 31/4709; C12Q 1/68; A61K 45/06; G01N 33/68; G01N 33/574

(52) U.S. Cl. CPC A61K 31/4709; G01N 33/6863; G01N 33/57407; A61K 45/06; C12Q 1/6886; C12Q 2600/158

(57) ABSTRACT

The present invention relates to the field of molecular biology, cell biology, and cancer biology. Specifically, the present invention relates to methods of treating myelodysplastic syndrome (“MDS”) with a farnesyltransferase inhibitor (FTI) that include determining whether the subject is likely to be responsive to the FTI treatment based on the Th1/Th2 balance and additional characteristics.

### CROSS-REFERENCE TO RELATED APPLICATIONS

This application claims the benefit of priority to U.S. Ser. No. 62/334,322 filed May 10, 2016, which is incorporated herein by reference in its entirety.

### FIELD

The present invention relates to the field of molecular biology, cell biology and cancer biology.

### BACKGROUND

Stratification of patient populations to improve therapeutic response rate is increasingly valuable in the clinical management of cancer patients. Farnesyltransferase inhibitors (FTI) are therapeutic agents that have utility in the treatment of cancers, such as Myelodysplastic syndrome (“MDS”). However, patients respond differently to an FTI treatment. Therefore, methods to predict the responsiveness of a subject having MDS to an FTI treatment, or methods to select MDS patients for an FTI treatment represent unmet needs. The methods and compositions of the present invention meet these needs and provide other related advantages.

### SUMMARY OF THE INVENTION

Provided herein are methods to treat MDS in a subject including administering a therapeutically effective amount of an FTI to the subject characterized by Th1 dominance. Provided herein are also methods to predict the responsiveness of a subject having MDS for an FTI treatment, methods to select a MDS patient for an FTI treatment, methods to stratify MDS patients for an FTI treatment, and methods to increase the responsiveness of a MDS patient population for an FTI treatment. In some embodiments, the methods include analyzing a sample from the subject having MDS to determining that the Th1/Th2 balance prior to administering the FTI to the subject. The methods can further include administering a therapeutically effective amount of an FTI to the subject characterized by Th1 dominance. In some embodiments, the MDS can be lower risk MDS. In some embodiments, the FTI is tipifarnib.

In some embodiments, the sample from the subject can be a tumor biopsy or a body fluid sample. In some embodiments, the sample can be a whole blood sample, a partially purified blood sample, a peripheral blood sample, a serum sample, a cell sample or a lymph node sample. In some embodiments, the sample can be peripheral blood mononuclear cells (PBMC).

In some embodiments, the methods provided herein include determining the expression level of a gene signature for Th1 cells in a sample from a subject having MDS, wherein the subject is determined to be characterized by Th1 dominance if the expression level in the sample is higher than a reference level of the gene signature.

The gene signatures for Th1 cells can be selected from the group consisting of TBX21, STAT1, STAT6, CXCR3, CCR5, IFN-γ, TNF-α, IL-2, IL-12, and any combination thereof. In some embodiments, methods provided herein include determining the expression levels of at least two, three, four, five, six, seven, eight, or nine gene signatures for Th1 cells.

In some embodiments, the methods provided herein include determining the expression level of TBX21 in a sample from a subject having MDS, and administering a therapeutically effective amount of an FTI to the subject if the TBX21 expression level in the sample is higher than a reference level of TBX21.

In some embodiments, the methods provided herein further include determining the expression level of a gene signature for Th2 cells in the sample from the subject having MDS, and the ratio of the expression level of a Th1 gene signature to that of the Th2 gene signature, wherein the subject is determined to be characterized by Th1 dominance if the ratio is higher than a reference ratio. The gene signature for Th2 cells can be GATA3, CCR4, IL-4, IL-5, IL-6, IL-10, IL-13, or any combination thereof. In some embodiments, the methods include determining the expression level of GATA3 in a sample from a subject having MDS.

In some embodiments, provided herein are methods to treat MDS that include determining the expression levels of TBX21 and GATA3 in a sample from a subject having MDS, and administering an therapeutically effective amount of an FTI to the subject if the ratio of the TBX21 expression level to GATA3 expression level is higher than a reference ratio.

In some embodiments, the methods provided herein include determining the mRNA level of a gene signature a sample from a subject having MDS. In some embodiments, the mRNA level of the gene signature is determined by Polymerase Chain Reaction (PCR), qPCR, qRT-PCR, RNA-seq, microarray analysis, SAGE, MassARRAY technique, next-generation sequencing, or FISH.

In some embodiments, the methods provided herein include determining the protein level of a gene signature a sample from a subject having MDS. In some embodiments, the protein level of the gene signature can be determined by an immunehistochemistry (IHC) assay, an immunoblotting (IB) assay, an immunofluorescence (IF) assay, flow cytometry (FACS), or an Enzyme-Linked Immunosorbent Assay (ELISA). The IHC assay can be H&E staining.

In some embodiments, the methods provided herein include analyzing cell constitution in a sample from the subject having MDS, wherein the subject is determined to be characterized by Th1 dominance if the sample has more Th1 cells than Th2 cells. In some embodiments, the methods provided herein include administering a therapeutically effective amount of an FTI to a subject having MDS if a sample from the subject has more Th1 cells than Th2 cells.

In some embodiments, the methods provided herein include determining the ratio of Th1 cells to Th2 cells in the sample from the subject having MDS to be higher than a reference ratio. In some embodiments, the reference ratio can be 1, 2, 3, 4, 5, 6, 7, 8, 9, 10, 20, 30, 40, 50, 75, or 100. In some embodiments, at least 20% of the cells in the sample from the subject having MDS are Th1 cells. In some embodiments, the methods provided herein include determining that at least 20%, at least 30%, at least 40%, at least 50%, at least 60%, at least 70%, at least 80%, or at least 90% of the cells in the sample are Th1 cells.

In some embodiments, the Th1 cells in the sample are identified by an IHC assay or FACS.

In some embodiments, the methods provided herein include analyzing cytokine profile in a sample from the subject having MDS, wherein the subject is determined to be characterized by Th1 dominance if a Th1 cytokine (namely, Th1 cell related cytokine) is detected in the sample. In some embodiments, the methods provided herein include administering a therapeutically effective amount of an FTI to a subject having MDS if a Th1 cytokine is detected in a sample from the subject. The Th1 cytokine can be IFN-γ, TNF-α, IL-2, IL-12, or any combination thereof. In some embodiments, the Th1 cytokine can be IFN-γ.

In some embodiments, the methods further include analyzing a Th2 cytokine (namely, Th2 cell related cytokine) in the sample, wherein the subject is determined to be characterized by Th1 dominance if the Th2 cytokine is absent in the sample. In some embodiments, the methods provided herein include administering a therapeutically effective amount of an FTI to a subject having MDS if a Th2 cytokine is not detected in a sample from the subject. The Th2 cytokine can be IL-4, IL5, IL-13, or any combination thereof. In some embodiments, the Th2 cytokine can be IL-4.

In some embodiments, the methods provided herein further include determining the level of the Th1 cytokine in a sample from a subject having MDS, wherein the subject is determined to be characterized by Th1 dominance if the level of the Th1 cytokine in the sample is higher than a reference level. In some embodiments, the methods provided herein include administering a therapeutically effective amount of an FTI to a subject having MDS if the level of a Th1 cytokine in a sample from the subject is higher than a reference level. The Th1 cytokine can be IFN-γ, TNF-α, IL-2, IL-12, or any combination thereof. In some embodiments, the Th1 cytokine can be IFN-γ.

In some embodiments, the methods provided herein further include determining the level of a Th2 cytokine in the sample, wherein the subject is determined to be characterized by Th1 dominance if the level of the Th2 cytokine is lower than a reference level. In some embodiments, the methods provided herein include administering a therapeutically effective amount of an FTI to a subject having MDS if the level of a Th2 cytokine in a sample from the subject is lower than a reference level. The Th2 cytokine can be IL-4, IL5, IL-13, or any combination thereof. In some embodiments, the Th2 cytokine can be IL-4.

In some embodiments, the methods provided herein further include determining the ratio of the level of a Th1 cytokine to that of a Th2 cytokine in a sample from a subject having MDS, and wherein the subject is determined to be characterized by Th1 dominance if the ratio is higher than a reference ratio. In some embodiments, the methods provided herein include administering a therapeutically effective amount of an FTI to a subject having MDS if the ratio of the level of a Th1 cytokine to that of a Th2 cytokine in a sample from the subject is higher than a reference ratio. The Th1 cytokine can be IFN-γ, TNF-α, IL-2, IL-12, or any combination thereof. The Th2 cytokine can be IL-4, IL5, IL-13, or any combination thereof. In some embodiments, the Th1 cytokine is IFN-γ and the Th2 cytokine is IL-4.

In some embodiments, the methods provided herein include analyzing cytokine profile in a sample from a subject having MDS by RT-PCR, microarray, Cytometric Bead Array, ELISA or Intracellular cytokine staining (ICS). In some embodiments, the sample is a serum sample.

In some embodiments, the methods provided herein to treat MDS in a subject characterized by Th1 dominance with an FTI, methods to predict the responsiveness of a subject having MDS for an FTI treatment, methods to select a MDS patient for an FTI treatment, methods to stratify MDS patients for an FTI treatment, and methods to increase the responsiveness of a MDS patient population for an FTI treatment further include determining the expression level of an additional gene signature selected from the group consisting of GNLY, PRF, GRMK, GZMH, GZMM, LYZ, CD8β, KIR2DS2, KIR2DS5, KIR3DL1 and KIR3DL2, in a sample from a subject having MDS, wherein if the expression level of the additional gene signature in the sample is higher than a reference expression level, the subject is predicted to be likely responsive to an FTI treatment, or is administered an therapeutically effective amount of an FTI.

In some embodiments, the additional gene signature is KIR2DS2, KIR2DS5, GZMM, or any combination thereof.

In some embodiments, the methods provided herein further include determining the ratio of the expression level of KIR2DS2 to the expression level of KIR2DL2 (the “KIR2DS2/KIR2DL2 ratio”) in a sample from a subject having MDS, wherein the subject is predicted to be likely responsive to an FTI treatment, or is administered an therapeutically effective amount of an FTI, if the KIR2DS2/KIR2DL2 ratio in the sample is higher than a reference ratio. In some embodiments, the methods provided herein further include determining the ratio of the expression level of KIR2DS5 to the expression level of KIR2DL5 (the “KIR2DS5/KIR2DL5 ratio”) in a sample from a subject having MDS, wherein the subject is predicted to be likely responsive to an FTI treatment, or is administered an therapeutically effective amount of an FTI, if the KIR2DS5/KIR2DL5 ratio in the sample is higher than a reference ratio.

In some embodiments, the methods provided herein further include determining the expression level of RASGRP1 in a sample from a subject having MDS. In some embodiments, a subject having MDS is predicted to be likely responsive to an FTI treatment, or is administered a therapeutically effective amount of an FTI if the expression level of RASGRP1 in a sample from the subject is higher than a reference expression level.

In some embodiments, the methods provided herein further include determining the mutation status of RhoA in a sample from a subject having MDS. In some embodiments, a subject having MDS is predicted to be likely responsive to an FTI treatment, or is administered a therapeutically effective amount of an FTI if the sample does not have RhoA mutation.

In some embodiments, the FTI is selected from the group consisting of tipifarnib, lonafarnib, CP-609,754, BMS-214662, L778123, L744823, L739749, R208176, AZD3409 and FTI-277. In some embodiments, the FTI is administered at a dose of 1-1000 mg/kg body weight. In some embodiments, the FTI is tipifarnib. In some embodiments, an FTI is administered at a dose of 200-1200 mg twice a day (“b.i.d.”). In some embodiments, an FTI is administered at a dose of 600 mg twice a day. In some embodiments, an FTI is administered at a dose of 900 mg twice a day. In some embodiments, an FTI is administered at a dose of 1200 mg twice a day. In some embodiments, an FTI is administered daily for a period of one to seven days. In some embodiments, an FTI is administered in alternate weeks. In some embodiments, an FTI is administered on days 1-7 and 15-21 of a 28-day treatment cycle. In some embodiments, the treatment period can continue for up to 12 months. In some embodiments, tipifarnib is administered orally at a dose of 900 mg twice a day on days 1-7 and 15-21 of a 28-day treatment cycle.

In some embodiments, an FTI is administered before, during, or after irradiation. In some embodiments, the methods provided herein also include administering a therapeutically effective amount of a secondary active agent or a support care therapy to the subject. In some embodiments, the secondary active agent is a DNA-hypomethylating agent, a therapeutic antibody that specifically binds to a cancer antigen, a hematopoietic growth factor, cytokine, anti-cancer agent, antibiotic, cox-2 inhibitor, immunomodulatory agent, anti-thymocyte globulin, immunosuppressive agent, corticosteroid or a pharmacologically derivative thereof. In some embodiments, the secondary active agent is a DNA-hypomethylating agent, such as azacitidine or decitabine.

### BRIEF DESCRIPTION OF THE DRAWINGS

FIG. 1 shows the Flow Cytometry data from 10 patients enrolled in a clinical study of Tipifarnib with a diagnosis of lower risk myelodysplastic syndrome. As shown, these patients were indicated Th1 or Th1/17 dominance prior to trial treatments, with five patients indicated with Th1/17 dominance, and five indicated with Th1 dominance.

FIG. 2 shows analysis of serum cytokine levels in lymphoma patients enrolled in a clinical study of Tipifarnib. As shown, tipifarnib induced downregulation of Th1 cytokine TNF-alpha production in subjects with high TNF-alpha levels.

### DETAILED DESCRIPTION

As used herein, the articles “a,” “an,” and “the” refer to one or to more than one of the grammatical object of the article. By way of example, a sample refers to one sample or two or more samples.

As used herein, the term “subject” refers to a mammal. A subject can be a human or a non-human mammal such as a dog, cat, bovid, equine, mouse, rat, rabbit, or transgenic species thereof. The subject can be a patient, a cancer patient, or a MDS cancer patient.

As used herein, the term “sample” refers to a material or mixture of materials containing one or more components of interest. A sample from a subject refers to a sample obtained from the subject, including samples of biological tissue or fluid origin, obtained, reached, or collected in vivo or in situ. A sample can be obtained from a region of a subject containing precancerous or cancer cells or tissues. Such samples can be, but are not limited to, organs, tissues, fractions and cells isolated from a mammal. Exemplary samples include lymph node, whole blood, partially purified blood, serum, bone marrow, and peripheral blood mononuclear cells (“PBMC”). A sample also can be a tissue biopsy. Exemplary samples also include cell lysate, a cell culture, a cell line, a tissue, oral tissue, gastrointestinal tissue, an organ, an organelle, a biological fluid, a blood sample, a urine sample, a skin sample, and the like.

As used herein, the term “analyzing” a sample refers to carrying that an art-recognized assay to make an assessment regarding a particular property or characteristic of the sample. The property or characteristic of the sample can be, for example, the type of the cells in the sample, or the expression level of a gene in the sample.

As used herein, the terms “treat,” “treating,” and “treatment,” when used in reference to a cancer patient, refer to an action that reduces the severity of the cancer, or retards or slows the progression of the cancer, including (a) inhibiting the cancer growth, or arresting development of the cancer, and (b) causing regression of the cancer, or delaying or minimizing one or more symptoms associated with the presence of the cancer.

As used herein, the term “administer,” “administering,” or “administration” refers to the act of delivering, or causing to be delivered, a compound or a pharmaceutical composition to the body of a subject by a method described herein or otherwise known in the art. Administering a compound or a pharmaceutical composition includes prescribing a compound or a pharmaceutical composition to be delivered into the body of a patient. Exemplary forms of administration include oral dosage forms, such as tablets, capsules, syrups, suspensions; injectable dosage forms, such as intravenous (IV), intramuscular (IM), or intraperitoneal (IP); transdermal dosage forms, including creams, jellies, powders, or patches; buccal dosage forms; inhalation powders, sprays, suspensions, and rectal suppositories.

As used herein, the term “therapeutically effective amount” of a compound when used in connection with a disease or disorder refers to an amount sufficient to provide a therapeutic benefit in the treatment or management of the disease or disorder or to delay or minimize one or more symptoms associated with the disease or disorder. A therapeutically effective amount of a compound means an amount of the compound that when used alone or in combination with other therapies, would provide a therapeutic benefit in the treatment or management of the disease or disorder. The term encompasses an amount that improves overall therapy, reduces or avoids symptoms, or enhances the therapeutic efficacy of another therapeutic agent. The term also refers to the amount of a compound that sufficiently elicits the biological or medical response of a biological molecule (e.g., a protein, enzyme, RNA, or DNA), cell, tissue, system, animal, or human, which is being sought by a researcher, veterinarian, medical doctor, or clinician.

As used herein, the term “gene signature” refers to a gene that is differentially expressed in different cell types, and whose expression level in a cell or in a sample can indicate the type of the cell or the cell constitution of the sample. For example, gene signatures for Th1 include, e.g., TBX21, STAT1, STAT6, CXCR3, CCR5, IFN-γ, TNF-α, IL-2, and IL-12, which are relatively highly expressed in Th1 cells as compared to other cell types, such as Th2 cells or naive CD4+ T cells. For example, gene signatures for Th2 include, e.g., GATA3, CCR4, IL-4, IL-5, IL-6, IL-10, and IL-13, which are relatively highly expressed in Th2 cells as compared to other cell types, such as Th1 cells or naive CD4+ T cells. The expression level of a gene signature for a particular cell type in a cell with undetermined origin, if higher than a reference level, can indicate the cell with undetermined origin is of this particular cell type. For example, the expression level of a gene signature for Th1 (e.g. TBX21) in a cell with undetermined origin, if higher than a reference level, can indicate that the cell is a Th1 cell. The expression level of a gene signature for Th1 (e.g. TBX21) in a sample with a cell population having undetermined origin can indicate the percentage of Th1 cells in the cell population.

As used herein, the term “express” or “expression” when used in connection with a gene refers to the process by which the information carried by the gene becomes manifest as the phenotype, including transcription of the gene to a messenger RNA (mRNA), the subsequent translation of the mRNA molecule to a polypeptide chain and its assembly into the ultimate protein.

As used herein, the term “expression level” of a gene refers to the amount or accumulation of the expression product of the gene, such as, for example, the amount of a RNA product of the gene (the RNA level of the gene) or the amount of a protein product of the gene (the protein level of the gene). The gene can be a gene signature associated with a particular cell type. If the gene has more than one allele, the expression level of a gene refers to the total amount of accumulation of the expression product of all existing alleles for this gene, unless otherwise specified. For example, the expression level of KIR2DL5 refers to the total expression levels of both KIR2DL5A and KIR2DL5B, unless otherwise specified.

As used herein, the term “reference” when used in connection with a quantifiable value refers to a predetermined value that one can use to determine the significance of the value as measured in a sample.

As used herein, the term “reference expression level” refers to a predetermined expression level of a gene that one can use to determine the significance of the expression level of the gene in a cell or in a sample. The gene can be a gene signature associated with a particular cell type. A reference expression level of a gene can be the expression level of the gene in a reference cell determined by a person of ordinary skill in the art. For example, the reference expression level of a gene signature for Th1 cells (e.g. TBX21) can be its average expression level in naive CD4+ T cells. Accordingly, one can determine the expression level of a gene signature for Th1 cells (e.g. TBX21) in a cell with undetermined origin, which, if higher than the average expression level of the gene signature in naive CD4+ T cells, indicates that the cell is a Th1 cell. A reference expression level of a gene can also be a cut-off value determined by a person of ordinary skill in the art through statistical analysis of the expression levels of the gene in various sample cell populations. For example, by analyzing the expression levels of TBX21 in sample cell populations having at least 50%, at least 60%, at least 70%, at least 80%, at least 90% Th1 cells, a person of ordinary skill in the art can determine a cut-off value as the reference expression level of TBX21, which can be used to indicate the percentages of Th1 cells in a cell population with unknown constitution. For example, a reference expression level of TBX21 can be predetermined by a person of ordinary skill in the art by the analysis mentioned above, and accordingly, one can then determine the expression level of TBX21 in a cell population with unknown constitution, which, if higher than the predetermined reference expression level, can indicate that the cell population has, for example, at least 50% (60%, 70%, 80%, or 90%) Th1 cells.

The term “reference ratio” as used herein in connection with the expression levels of two genes refers to a ratio predetermined by a person of ordinary skill in the art that can be used to determine the significance of the ratio of the levels of these two genes in a cell or in a sample. The two genes can be two gene signatures associated with two different cell types. The reference ratio of the expression levels of two genes can be the ratio of expression levels of these two genes in a reference cell determined by a person of ordinary skill in the art. For example, the reference ratio of expression levels of TBX21 (a gene signature of Th1 cells) and GATA3 (a gene signature for Th2 cells) can be the average ratio of the expression levels of these two genes in a naive CD4+ T cells. As such, the ratio of the expression levels of these two genes in a cell with undetermined origin, if higher than the reference ratio, indicates that the cell is a Th1 cell. A reference ratio can also be a cut-off value determined by a person of ordinary skill in the art through statistical analysis of ratios of expression levels of the two genes in various sample cell populations. For example, by analyzing the ratios of expression level of TBX21 to that of GATA3 in sample cell populations having at least 50%, at least 60%, at least 70%, at least 80%, at least 90% Th1 cells, a person of ordinary skill in the art can determine a cut-off value as the reference ratio, which can be used to indicate the percentages of Th1 cells in a cell population with unknown constitution. For example, a reference ratio can be predetermined by a person of ordinary skill in the art by the analysis mentioned above, and accordingly, the ratio of expression level of TBX21 to the expression level of GATA3 in a cell population with unknown constitution, if higher than the predetermined reference ratio, can indicate that the cell population has, for example, at least 50% (60%, 70%, 80%, or 90%) Th1 cells.

As used herein, the term “responsiveness” or “responsive” when used in connection with a treatment refers to the effectiveness of the treatment in lessening or decreasing the symptoms of the disease being treated. For example, a cancer patient is responsive to an FTI treatment if the FTI treatment effectively inhibits the cancer growth, or arrests development of the cancer, causes regression of the cancer, or delays or minimizes one or more symptoms associated with the presence of the cancer in this patient.

The responsiveness to a particular treatment of a cancer patient can be characterized as a complete or partial response. “Complete response” or “CR” refers to an absence of clinically detectable disease with normalization of previously abnormal radiographic studies, lymph node, and cerebrospinal fluid (CSF) or abnormal monoclonal protein measurements. “Partial response,” or “PR,” refers to at least about a 10%, 20%, 30%, 40%, 50%, 60%, 70%, 80%, or 90% decrease in all measurable tumor burden (i.e., the number of malignant cells present in the subject, or the measured bulk of tumor masses or the quantity of abnormal monoclonal protein) in the absence of new lesions.

A person of ordinary skill in the art would understand that clinical standards used to define CR, PR, or other level of patient responsiveness to treatments can vary for different subtypes of cancer. For example, for hematopoietic cancers, patient being “responsive” to a particular treatment can be defined as patients who have a complete response (CR), a partial response (PR), or hematological improvement (HI) (Lancet et al., Blood 2:2 (2006)). HI can be defined as any lymph node blast count less than 5% or a reduction in lymph node blasts by at least half. On the other hand, patient being “not responsive” to a particular treatment can be defined as patients who have either progressive disease (PD) or stable disease (SD). Progressive disease (PD) can be defined as either >50% increase in lymph node or circulating blast % from baseline, or new appearance of circulating blasts (on at least 2 consecutive occasions). Stable disease (SD) can be defined as any response not meeting CR, PR, HI, or PD criteria.

As used herein, the term “likelihood” refers to the probability of an event. A subject is “likely” to be responsive to a particular treatment when a condition is met means that the probability of the subject to be responsive to a particular treatment is higher when the condition is met than when the condition is not met. The probability to be responsive to a particular treatment can be higher by, for example, 5%, 10%, 25%, 50%, 100%, 200%, or more in a subject who meets a particular condition compared to a subject who does not meet the condition. For example, a subject having MDS is “likely” responsive to an FTI treatment when the subject is characterized by Th1 dominance means that the probability of a subject to be responsive to FTI treatment is 5%, 10%, 25%, 50%, 100%, 200%, or more higher in a subject who is characterized by Th1 dominance compared to a subject who is not. For another example, a subject is “likely” responsive to tipifarnib treatment when the expression level of TBX21 in a sample from the subject is higher than a reference expression level of TBX21 means that the probability of a subject to be responsive to tipifarnib treatment is 5%, 10%, 25%, 50%, 100%, 200%, or more higher in a subject whose sample has a higher the expression level of TBX21 than a reference expression level compared to a subject whose expression level of TBX21 is not higher than the same reference expression level.

MDS refer to a group of blood and bone marrow disorders with both proliferative and dysplastic phenotypes. MDS can be characterized by a cellular marrow with impaired morphology and maturation (dysmyelopoiesis), ineffective blood cell production, or hematopoiesis, leading to low blood cell counts, or cytopenias, and high risk of progression to acute myeloid leukemia, resulting from ineffective blood cell production. See The Merck Manual 953 (17th ed. 1999) and List et al., 1990, J Clin. Oncol. 8:1424.

MDS can be divided into a number of subtypes depending on at least 1) whether increased numbers of blast cells are present in bone marrow or blood, and what percentage of the marrow or blood is made up of these blasts; 2) whether the marrow shows abnormal growth (dysplasia) in only one type of blood cell (unilineage dysplasia) or in more than one type of blood cell (multilineage dysplasia); and 3) whether there are chromosome abnormalities in marrow cells and, if so, which type or types of abnormalities. MDS can also categorized based on the surface markers of the cancer cells. According to the World Health Organization, MDS subtypes include refractory cytopenia with unilineage dysplasia (RCUD), also known as refractory anemia, refractory neutropenia, or refractory thrombocytopenia; refractory anemia with ring sideroblasts (RARS); refractory cytopenia with multilineage dysplasia (RCMD), which includes RCMD-RS if multilineage dysplasia and ring sideroblasts both are present; refractory anemia with excess blasts-1 (RAEB-1) and refractory anemia with excess blasts-2 (RAEB-2) (These subtypes mean that the patients have at least 5 percent (RAEB-1) or at least 10 percent (RAEB-2) but less than 20 percent blasts in their marrow); MDS associated with isolated abnormality of chromosome 5 [del(5q)]; and unclassifiable MDS (MDS-U).

As a group of hematopoietic stem cell malignancies with significant morbidity and mortality, MDS is a highly heterogeneous disease, and the severity of symptoms and disease progression can vary widely among patients. The current standard clinical tool to evaluate risk stratification and treatment options is the revised International Prognostic Scoring System, or IPSS-R. The IPSS-R differentiates patients into five risk groups (Very Low, Low, Intermediate, High, Very High) based on evaluation of cytogenetics, percentage of blasts (undifferentiated blood cells) in the bone marrow, hemoglobin levels, and platelet and neutrophil counts. The WHO also suggested stratifying MDS patients by a del (5q) abnormality.

According to the ACS, the annual incidence of MDS is approximately 13,000 patients in the United States, the majority of which are 60 years of age or older. Approximately 75% of patients (about 10,000) fall into the IPSS-R risk categories of Very Low, Low, and Intermediate, or collectively known as lower risk MDS. Autoimmunity can play a role in the onset of lower risk MDS.

The initial hematopoietic stem cell injury can be from causes such as, but not limited to, cytotoxic chemotherapy, radiation, virus, chemical exposure, and genetic predisposition. A clonal mutation predominates over bone marrow, suppressing healthy stem cells. In the early stages of MDS, the main cause of cytopenias is increased programmed cell death (apoptosis). As the disease progresses and converts into leukemia, gene mutation rarely occurs and a proliferation of leukemic cells overwhelms the healthy marrow. The disease course differs, with some cases behaving as an indolent disease and others behaving aggressively with a very short clinical course that converts into an acute form of leukemia.

An international group of hematologists, the French-American-British (FAB) Cooperative Group, classified MDS disorders into five subgroups, differentiating them from AML. The Merck Manual 954 (17^{th} ed. 1999); Bennett J. M., et al., Ann. Intern. Med. 1985 October, 103(4): 620-5; and Besa E. C., Med. Clin. North Am. 1992 May, 76(3): 599-617. An underlying trilineage dysplastic change in the bone marrow cells of the patients is found in all subtypes.

There are two subgroups of refractory anemia characterized by five percent or less myeloblasts in bone marrow: (1) refractory anemia (RA) and; (2) RA with ringed sideroblasts (RARS), defined morphologically as having 15% erythroid cells with abnormal ringed sideroblasts, reflecting an abnormal iron accumulation in the mitochondria. Both have a prolonged clinical course and low incidence of progression to acute leukemia. Besa E. C., Med. Clin. North Am. 1992 May, 76(3): 599-617.

There are two subgroups of refractory anemias with greater than five percent mycloblasts: (1) RA with excess blasts (RAEB), defined as 6-20% myeloblasts, and (2) RAEB in transformation (RAEB-T), with 21-30% myeloblasts. The higher the percentage of myeloblasts, the shorter the clinical course and the closer the disease is to acute myelogenous leukemia. Patient transition from early to more advanced stages indicates that these subtypes are merely stages of disease rather than distinct entities. Elderly patients with MDS with trilineage dysplasia and greater than 30% myeloblasts who progress to acute leukemia are often considered to have a poor prognosis because their response rate to chemotherapy is lower than de novo acute myeloid leukemia patients. The fifth type of MDS, the most difficult to classify, is CMML. This subtype can have any percentage of myeloblasts but presents with a monocytosis of 1000/dL or more. It may be associated with splenomegaly. This subtype overlaps with a myeloproliferative disorder and may have an intermediate clinical course. It is differentiated from the classic CML that is characterized by a negative Ph chromosome.

MDS is primarily a disease of elderly people, with the median onset in the seventh decade of life. The median age of these patients is 70-75 years, with ages ranging from the early third decade of life to as old as 80 years or older. The syndrome may occur in any age group, including the pediatric population. 75% of MDS patients are at high risk of infection, require regular transfusions and have a generally poor quality of life. About 25% of MDS patients transform to AML.

Patients who survive malignancy treatment with alkylating agents, with or without radiotherapy, have a high incidence of developing MDS or secondary acute leukemia. About 60-70% of patients do not have an obvious exposure or cause for MDS, and are classified as primary MDS patients.

The treatment of MDS is based on the stage and the mechanism of the disease that predominates the particular phase of the disease process. Bone marrow transplantation has been used in patients with poor prognosis or late-stage MDS. Epstein and Slease, 1985, Surg. Ann. 17:125. An alternative approach to therapy for MDS is the use of hematopoietic growth factors or cytokines to stimulate blood cell development in a recipient. Dexter, 1987, J. Cell Sci. 88:1; Moore, 1991, Annu. Rev. Immunol. 9:159; and Besa E. C., Med. Clin. North Am. 1992 May, 76(3): 599-617. The treatment of MDS using immunomodulatory compounds is described in U.S. Pat. No. 7,189,740, the entirety of which is hereby incorporated by reference.

Therapeutic options fall into three categories including supportive care, low intensity and high intensity therapy. Supportive care includes the use red blood cell and platelet transfusions and hematopoietic cytokines such as erythropoiesis stimulating agents or colony stimulating factors to improve blood counts. Low intensity therapies include hypomethylating agents such as azacytidine (Vidaza®) and decitabine (Dacogen®), biological response modifiers such as lenalidomide (Revlimid®), and immunosuppressive treatments such as cyclosporine A or antithymocyte globulin. High intensity therapies include chemotherapeutic agents such as idarubicin, azacytidine, fludarabine and topotecan, and hematopoietic stem cell transplants, or HSCT.

National Comprehensive Cancer Network, or NCCN, guidelines recommend that lower risk patients (IPSS-R groups Very Low, Low, Intermediate) receive supportive care or low intensity therapies with the major therapeutic goal of hematologic improvement, or HI. NCCN guidelines recommend that higher risk patients (IPSS-R groups High, Very High) receive more aggressive treatment with high intensity therapies. In some cases, high risk patients are unable to tolerate chemotherapy, and may elect lower intensity regimens. Despite currently available treatments, a substantial portion of MDS patients lack effective therapies and NCCN guidelines recommend clinical trials as additional therapeutic options. Treatment of MDS remains a significant unmet need requiring the development of novel therapies.

T cells can be separated into three major groups based on function: cytotoxic T cells, helper T cells (Th), and regulatory T cells (Tregs). Differential expression of markers on the cell surface, as well as their distinct cytokine secretion profiles, provide valuable clues to the diverse nature and function of T cells. For example, CD8+ cytotoxic T cells destroy infected target cells through the release of perforin, granzymes, and granulysin, whereas CD4+T helper cells have little cytotoxic activity and secrete cytokines that act on other leucocytes such as B cells, macrophages, eosinophils, or neutrophils to clear pathogens. Tregs suppress T-cell function by several mechanisms including binding to effector T-cell subsets and preventing secretion of their cytokines.

Helper T cells can be further categorized into difference classes, including e.g., Th1, Th2, Th9, Th17, and Tfh cells. Th17 cells give rise to Th1 cells. The hybrid Th1/17 cells exhibit a combinational phenotype of Th1 and Th17 subsets and share same gene signatures with Th1 cells, such as CXCR3. As used herein, Th1/17 cells are considered a subpopulation of Th1 cells, and Th1 dominance includes Th1/17 dominance. Differentiation of CD4+ T cells into Th1 and Th2 effector cells is largely controlled by the transcription factors TBX21 (T-Box Protein 21; T-bet) and GATA3 (GATA3), respectively. Both TBX21 and GATA3 are transcription factors that are master regulators of gene expression profiles in T helper (Th) cells, skewing Th polarization into Th1 and Th2 differentiation pathways, respectively. Thus, Th1 cells are characterized by high expression levels of TBX21 and the target genes activated by TBX21, and low expression levels of GATA3 and genes activated by GATA3. To the contrary, Th2 cells are characterized by high expression levels of GATA3 and the target genes activated by GATA3, and low expression levels of TBX21 and genes activated by TBX21.

An exemplary amino acid sequence and a corresponding encoding nucleic acid sequence of human TBX21 (GENBANK: NM 013351.1 GI:7019548) are provided below:

(SEQ ID NO: 1)
MGIVEPGCGDMLTGTEPMPGSDEGRAPGADPQHRYFYPEPGAQDADERRGG
GSLGSPYPGGALVPAPPSRFLGAYAYPPRPQAAGFPGAGESFPPPADAEGY
QPGEGYAAPDPRAGLYPGPREDYALPAGLEVSGKLRVALNNHLLWSKFNQH
QTEMIITKQGRRMFPFLSFTVAGLEPTSHYRMFVDVVLVDQHHWRYQSGKW
VQCGKAEGSMPGNRLYVHPDSPNTGAHWMRQEVSFGKLKLTNNKGASNNVT
QMIVLQSLHKYQPRLHIVEVNDGEPEAACNASNTHIFTFQETQFIAVTAYQ
NAEITQLKIDNNPFAKGFRENFESMYTSVDTSIPSPPGPNCQFLGGDHYSP
LLPNQYPVPSRFYPDLPGQAKDVVPQAYWLGAPRDHSYEAEFRAVSMKPAF
LPSAPGPTMSYYRGQEVLAPGAGWPVAPQYPPKMGPASWFRPMRTLPMEPG
PGGSEGRGPEDQGPPLVWTEIAPIRPESSDSGLGEGDSKRRRVSPYPSSG
DSSSPAGAPSPFDKEAEGQFYNYFPN
(SEQ ID NO: 2)
ATGGGCATCG TGGAGCCGGG TTGCGGAGAC ATGCTGACGG
GCACCGAGCC GATGCCGGGG AGCGACGAGG GCCGGGCGCC
TGGCGCCGAC CCGCAGCACC GCTACTTCTA CCCGGAGCCG
GGCGCGCAGG ACGCGGACGA GCGTCGCGGG GGCGGCAGCC
TGGGGTCTCC CTACCCGGGG GGCGCCTTGG TGCCCGCCCC
GCCGAGCCGC TTCCTTGGAG CCTACGCCTA CCCGCCGCGA
CCCCAGGCGG CCGGCTTCCC CGGCGCGGGC GAGTCCTTCC
CGCCGCCCGC GGACGCCGAG GGCTACCAGC CGGGCGAGGG
CTACGCCGCC CCGGACCCGC GCGCCGGGCT CTACCCGGGG
CCGCGTGAGG ACTACGCGCT ACCCGCGGGA CTGGAGGTGT
CGGGGAAACT GAGGGTCGCG CTCAACAACC ACCTGTTGTG
GTCCAAGTTT AATCAGCACC AGACAGAGAT GATCATCACC
AAGCAGGGAC GGCGGATGTT CCCATTCCTG TCATTTACTG
TGGCCGGGCT GGAGCCCACC AGCCACTACA GGATGTTTGT
GGACGTGGTC TTGGTGGACC AGCACCACTG GCGGTACCAG
AGCGGCAAGT GGGTGCAGTG TGGAAAGGCC GAGGGCAGCA
TGCCAGGAAA CCGCCTGTAC GTCCACCCGG ACTCCCCCAA
CACAGGAGCG CACTGGATGC GCCAGGAAGT TTCATTTGGG
AAACTAAAGC TCACAAACAA CAAGGGGGCG TCCAACAATG
TGACCCAGAT GATTGTGCTC CAGTCCCTCC ATAAGTACCA
GCCCCGGCTG CATATCGTTG AGGTGAACGA CGGAGAGCCA
GAGGCAGCCT GCAACGCTTC CAACACGCAT ATCTTTACTT
TCCAAGAAAC CCAGTTCATT GCCGTGACTG CCTACCAGAA
TGCCGAGATT ACTCAGCTGA AAATTGATAA TAACCCCTTT
GCCAAAGGAT TCCGGGAGAA CTTTGAGTCC ATGTACACAT
CTGTTGACAC CAGCATCCCC TCCCCGCCTG GACCCAACTG
TCAATTCCTT GGGGGAGATC ACTACTCTCC TCTCCTACCC
AACCAGTATC CTGTTCCCAG CCGCTTCTAC CCCGACCTTC
CTGGCCAGGC GAAGGATGTG GTTCCCCAGG CTTACTGGCT
GGGGGCCCCC CGGGACCACA GCTATGAGGC TGAGTTTCGA
GCAGTCAGCA TGAAGCCTGC ATTCTTGCCC TCTGCCCCTG
GGCCCACCAT GTCCTACTAC CGAGGCCAGG AGGTCCTGGC
ACCTGGAGCT GGCTGGCCTG TGGCACCCCA GTACCCTCCC
AAGATGGGCC CGGCCAGCTG GTTCCGCCCT ATGCGGACTC
TGCCCATGGA ACCCGGCCCT GGAGGCTCAG AGGGACGGGG
ACCAGAGGAC CAGGGTCCCC CCTTGGTGTG GACTGAGATT
GCCCCCATCC GGCCGGAATC CAGTGATTCA GGACTGGGCG
AAGGAGACTC TAAGAGGAGG CGCGTGTCCC CCTATCCTTC
CAGTGGTGAC AGCTCCTCCC CTGCTGGGGC CCCTTCTCCT
TTTGATAAGG AAGCTGAAGG ACAGTTTTAT AACTATTTTC
CCAACTGA

Thus, Th1 cells and Th2 cells can be classifieds by expressing either TBX21 and TBX21 activated genes (e.g. STAT1, STAT6, CXCR3, CCR5, IFN-γ, TNF-α, IL-2, IL-12) or GATA3 and GATA3 activated genes (e.g. CCR4, IL-4, IL-5, IL-6, IL-10, and IL-13). Th1 cells and Th2 cells can also be classifieds by, for example, expressing different proteins on the cell surface, otherwise known as having different surface profiles. For example, Th1 cells express CXCR3 on their surface, while Th2 cells express CCR4. Th1 cells and Th2 cells are also classified by, for example, secreting different specific cytokines in response to antigenic stimulation. Th1 cells primarily produce interferon (IFN)-γ, tumor necrosis factor (TNF)-α and interleukin (IL)-2, whereas Th2 cells primarily produce IL-4, IL-5, IL-6, IL-10, and IL-13. The cytokines produced by each Th subset tend to both stimulate production of that Th subset, and inhibit development of the other Th subset. For example, IFN-γ produced by Th1 cells has the dual effect of both stimulating Th1 development, and inhibiting Th2 development. Th2-secreted IL-10 has the opposite effect. The two helper T cell classes also differ by the type of immune response they produce. While Th1 cells tend to generate responses against intracellular parasites such as bacteria and viruses, Th2 cells produce immune responses against helminths and other extracellular parasites.

MDS patients can be categorized based on their Th1/Th2 balance. In particular, some MDS patients are characterized by Th1 dominance. Th1 dominance is characterized by a high ratio of Th1 to Th2 cells, and high expression of TBX21 (t-bet) and associated target genes, while the Th2 dominance is characterized by a high ratio of Th2 to Th1 cells, and high expression of GATA3 and associated target genes. The molecular signatures associated with high expression of these transcription factors were also enriched in other Th2- or Th1-associated transcripts. The sample from MDS pateints characterized by Th1 dominance (e.g. TBX21 overexpression) can also have a cytotoxic profile, characterized by expression of genes associated with cytotoxic activities such as GNLY, PRF, GRMK, GZMH, GZMM, LYZ, CD8β and KIR molecules including, for example, KIR2DS2, KIR2DS5, KIR3DL1 and KIR3DL2.

Provided herein are methods for selecting a subject having MDS for treatment with a FTI. The methods provided herein are based, in part, on the discovery that the patients having MDS characterized by Th1 dominance are more responsive to an FTI treatment, and that the clinical benefits of FTI is associated with the expression level of certain gene signatures associated with the Th1/Th2 balance in the MDS. Specifically, methods provided herein are based on the discovery that patients characterized by Th1 dominance are likely responsive to an FTI treatment, and selection of MDS patient population characterized by Th1 dominance for an FTI treatment can increase the overall response rate of the FTI treatment for MDS. In some embodiments, the MDS can be lower risk MDS. In some embodiments, the FTI is tipifarnib.

Accordingly, provided herein are methods for increasing the responsiveness of an FTI treatment for MDS by selectively treating MDS patients characterized by Th1 dominance. Provided herein are also methods for MDS patient population selection for an FTI treatment. Provided herein are also methods of predicting responsiveness of a subject having MDS to an FTI treatment based on the Th1/Th2 balance, wherein a subject is predicted to be likely response if the subject is characterized by Th1 dominance.

In some embodiments, provided herein are methods to treat MDS in a subject characterized by Th1 dominance, including administering a therapeutically effective amount of an FTI to the subject having MDS and characterized by Th1 dominance. In some embodiments, the methods include analyzing a sample from the subject having MDS to determine that the subject is characterized by Th1 dominance.

In some embodiments, methods provided herein also include obtaining a sample from the subject. The sample used in the methods provided herein includes body fluids from a subject or a tumour biopsy from the subject.

In some embodiments, the sample used in the present methods includes a biopsy (e.g., a tumor biopsy). The biopsy can be from any organ or tissue, for example, skin, liver, lung, heart, colon, kidney, bone marrow, teeth, lymph node, hair, spleen, brain, breast, or other organs. Any biopsy technique known by those skilled in the art can be used for isolating a sample from a subject, for instance, open biopsy, close biopsy, core biopsy, incisional biopsy, excisional biopsy, or fine needle aspiration biopsy. In some embodiments, the sample is a lymph node biopsy. In some embodiments, the sample can be a frozen tissue sample. In some embodiments, the sample can be a formalin-fixed paraffin-embedded (“FFPE”) tissue sample. In some embodiments, the sample can be a deparaffinised tissue section.

In some embodiments, the sample is a body fluid sample. Non-limiting examples of body fluids include blood (e.g., peripheral whole blood, peripheral blood), blood plasma, bone marrow, amniotic fluid, aqueous humor, bile, lymph, menses, serum, urine, cerebrospinal fluid surrounding the brain and the spinal cord, synovial fluid surrounding bone joints.

In some embodiments, the sample is a blood sample. The blood sample can be a whole blood sample, a partially purified blood sample, or a peripheral blood sample. The blood sample can be obtained using conventional techniques as described in, e.g. Innis et al, editors, PCR Protocols (Academic Press, 1990). White blood cells can be separated from blood samples using convention techniques or commercially available kits, e.g. RosetteSep kit (Stein Cell Technologies, Vancouver, Canada). Sub-populations of white blood cells, e.g. mononuclear cells, NK cells, B cells, T cells, monocytes, granulocytes or lymphocytes, can be further isolated using conventional techniques, e.g. magnetically activated cell sorting (MACS) (Miltenyi Biotec, Auburn, Calif.) or fluorescently activated cell sorting (FACS) (Becton Dickinson, San Jose, Calif.).

In one embodiment, the blood sample is from about 0.1 mL to about 10.0 mL, from about 0.2 mL to about 7 mL, from about 0.3 mL to about 5 mL, from about 0.4 mL to about 3.5 mL, or from about 0.5 mL to about 3 mL. In another embodiment, the blood sample is about 0.3, 0.4, 0.5, 0.6, 0.7, 0.8, 0.9, 1.0, 1.5, 2.0, 2.5, 3.0, 3.5, 4.0, 4.5, 5.0, 6.0, 7.0, 8.0, 9.0 or 10.0 mL.

In one embodiment, the sample is a bone marrow sample. Procedures to obtain a bone marrow sample are well known in the art, including but not limited to bone marrow biopsy and bone marrow aspiration. Bone marrow has a fluid portion and a more solid portion. In bone marrow biopsy, a sample of the solid portion is taken. In bone marrow aspiration, a sample of the fluid portion is taken. Bone marrow biopsy and bone marrow aspiration can be done at the same time and referred to as a bone marrow exam.

In certain embodiments, the sample used in the methods provided herein includes a plurality of cells. Such cells can include any type of cells, e.g., stem cells, blood cells (e.g., PBMCs), lymphocytes, NK cells, B cells, T cells, monocytes, granulocytes, immune cells, or tumor or cancer cells. Specific cell populations can be obtained using a combination of commercially available antibodies (e.g., Quest Diagnostic (San Juan Capistrano, Calif.); Dako (Denmark)). In certain embodiments, the sample used in the methods provided herein includes PBMCs.

In certain embodiments, the sample used in the methods provided herein includes a plurality of cells from the diseased tissue, for example, the MDS tumor sample from the subject. In some embodiments, the cells can be obtained from the tumor tissue, such as a tumor biopsy or a tumor explants. In certain embodiments, the number of cells used in the methods provided herein can range from a single cell to about 10⁹ cells. In some embodiments, the number of cells used in the methods provided herein is about 1×10⁴ 5×10⁴ 1×10⁵ 5×10⁵ 1×10⁶ 5×10⁶ 1×10⁷, 5×10⁷, 1×10⁸, or 5×10⁸.

The number and type of cells collected from a subject can be monitored, for example, by measuring changes in morphology and cell surface markers using standard cell detection techniques such as flow cytometry, cell sorting, immunocytochemistry (e.g., staining with tissue specific or cell-marker specific antibodies) fluorescence activated cell sorting (FACS), magnetic activated cell sorting (MACS), by examination of the morphology of cells using light or confocal microscopy, and/or by measuring changes in gene expression using techniques well known in the art, such as PCR and gene expression profiling. These techniques can be used, too, to identify cells that are positive for one or more particular markers. Fluorescence activated cell sorting (FACS) is a well-known method for separating particles, including cells, based on the fluorescent properties of the particles (Kamarch, 1987, Methods Enzymol, 151:150-165). Laser excitation of fluorescent moieties in the individual particles results in a small electrical charge allowing electromagnetic separation of positive and negative particles from a mixture. In one embodiment, cell surface marker-specific antibodies or ligands are labeled with distinct fluorescent labels. Cells are processed through the cell sorter, allowing separation of cells based on their ability to bind to the antibodies used. FACS sorted particles may be directly deposited into individual wells of 96-well or 384-well plates to facilitate separation and cloning.

In certain embodiments, subsets of cells are used in the methods provided herein. Methods to sort and isolate specific populations of cells are well-known in the art and can be based on cell size, morphology, or intracellular or extracellular markers. Such methods include, but are not limited to, flow cytometry, flow sorting, FACS, bead based separation such as magnetic cell sorting, size-based separation (e.g., a sieve, an array of obstacles, or a filter), sorting in a microfluidics device, antibody-based separation, sedimentation, affinity adsorption, affinity extraction, density gradient centrifugation, laser capture microdissection, etc.

In some embodiments, the sample used in methods provided herein can be a whole blood sample, a partially purified blood sample, a peripheral blood sample, a serum sample, a cell sample or a lymph node sample. The sample can be a tissue biopsy or a tumor biopsy. In some embodiments, the sample is a lymph node biopsy from a subject having MDS. In some embodiments, the sample is the PBMCs from a subject having MDS.

The Th1/Th2 balance can be determined by a variety of methods as described herein or otherwise known in the art. In some embodiments, the Th1 dominance of a subject having MDS is characterized by the relatively high expression level of one or more gene signatures associated with Th1 cells. Accordingly, provided herein are methods of selecting MDS patients for an FTI treatment, methods of predicating responsiveness of a subject having MDS to an FTI treatment, methods of increasing the responsiveness to an FTI treatment of a MDS patient population, based on the expression level of one or more gene signatures associated with Th1 cells (i.e. Th1 gene signature). In some embodiments, a subject having MDS is selected for an FTI treatment if the expression level of a Th1 gene signature in his or her sample is determined to be higher than a reference level of the Th1 signature gene.

In some embodiments, provided herein are methods of treating MDS in a subject characterized by Th1 dominance, including analyzing a sample from a subject having MDS to determine that the expression level of a gene signature for Th1 cells in the sample to be higher than a reference expression level of the gene signature, and administering a therapeutically effective amount of an FTI to the subject.

The sample can be a tumor biopsy, a blood sample, a lymph node sample, or any other sample disclosed herein. In some embodiments, the FTI is tipifarnib.

The gene signatures associated with the Th1 cells include the transcription factor TBX21 (T-bet) and the target genes that are activated by TBX21. In some embodiments, the gene signature includes for example, TBX21, STAT1 (Signal Transducer and Activator of Transcription 1), STAT6 (Signal Transducer and Activator of Transcription 6), CXCR3 (Chemokine (C-X-C Motif) Receptor 3; also known as CD183), CCR5 (Chemokine (C-C Motif) Receptor 5), IFN-γ (Interferon-gamma), TNF-α (Tumor Necrosis Factor-Alpha), IL-2 (Interleukin 2), and IL-12 (Interleukin 12). In some embodiments, the gene signature is TBX21. In some embodiments, the gene signature is STAT1. In some embodiments, the gene signature is CXCR3. In some embodiments, the gene signature CCR5. In some embodiments, the gene signature is IFN-γ. In some embodiments, the gene signature TNF-α. In some embodiments, the gene signature is IL-2. In some embodiments, the gene signature is IL-12.

As the Th1 dominance can also be characterized by the expression of a subset of genes associated with a cytotoxic profile, such as GNLY (Granulysin), PRF (Papillomavirus Regulatory Factor), GRMK (Granzyme K), GZMH (Granzyme K), GZMM (Granzyme M), LYZ (Lysozyme), CD8β (T-cell surface glycoprotein CD8 beta chain) and KIR (Killer Cell Immunoglobulin-Like Receptor) molecules including, for example, KIR2DS2, KIR2DS5, KIR3DL1 and KIR3DL2. Thus, methods provided herein can also include determining the expression level of one or more additional gene signatures selected from GNLY, PRF, GRMK, GZMH, GZMM, LYZ, CD8β, KIR2DS2, KIR2DS5, KIR3DL1 and KIR3DL2 in a sample from a subject having MDS, wherein a higher expression level of the additional gene signature compared to a reference expression level indicates that the subject is likely responsive to an FTI treatment. In some embodiments, the additional gene signature is GNLY. In some embodiments, the additional gene signature is PRF. In some embodiments, the additional gene signature is GRMK. In some embodiments, the additional gene signature is GZMH. In some embodiments, the additional gene signature is GZMM. In some embodiments, the additional gene signature is LYZ. In some embodiments, the additional gene signature is CD8β. In some embodiments, the additional gene signature is KIR2DS2. In some embodiments, the additional gene signature is KIR2DS5. In some embodiments, the additional gene signature is KIR3DL1. In some embodiments, the additional gene signature is KIR3DL2.

In some embodiments, the methods provided herein include determining the expression level of at least one gene signature for Th1 cells. In some embodiments, the methods provided herein include determining the expression level of at least one gene signature for Th1 cells. In some embodiments, the methods provided herein include determining the expression level of at least two gene signatures for Th1 cells. In some embodiments, the methods provided herein include determining the expression level of at least three gene signatures for Th1 cells. In some embodiments, the methods provided herein include determining the expression level of at least four, at least five, at least six, at least seven, at least eight, at least nine, at least ten gene signatures for Th1 cells. The higher expression levels of gene signatures for Th1 cells in a sample from a subject having MDS as compared to a reference level of the gene signature indicate the subject having MDS is likely responsive to an FTI treatment.

In some embodiments, the Th1 gene signature is TBX21.

In some embodiments, the Th1 gene signature is CXCR3.

In some embodiments, the methods provided herein further include determining the expression level of one or more gene signatures for Th2 cells, and the lower expression levels of gene signatures in a sample from a subject having MDS as compared to a reference level of the gene signature indicates that the subject having MDS is likely responsive to an FTI treatment. In some embodiments, a therapeutically effective amount of an FTI is administered to a subject having MDS if the expression level of a Th2 gene signature in a sample from the subject is lower than a reference level of the Th2 gene signature.

In some embodiments, the methods provided herein further include determining the ratio of the expression level of a Th1 gene signature to the expression level of a Th2 gene signature in the same sample. In some embodiments, a subject having MDS is predicted to be responsive to the FTI treatment if the ratio of the expression level of a Th1 gene signature to the expression level of a Th2 gene signature in a sample from the patient is higher than a reference ratio. In some embodiments, a therapeutically effective amount of an FTI is administered to a subject having MDS if the ratio of the expression level of a Th1 gene signature to the expression level of a Th2 gene signature in a sample from the patient is higher than a reference ratio.

The reference ratio can be determined by a person of ordinary skill in the art. The reference ratio can be the average ratio of the expression levels of the two gene signatures in a reference cell determined by a person of ordinary skill in the art, such as naive CD4+ cells. In some embodiments, the reference ratio can be 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 200, 300, 400, 500, 600, 700, 800, 900, or 1000.

The Th1 gene signatures include, for example, TBX21, STAT1, STAT6, CXCR3, CCR5, IFN-γ, TNF-α, IL-2, or IL-12. Th2 gene signatures include, for example, GATA3, CCR4, IL-4, IL-5, IL-6, IL-10, and IL-13. In some embodiments, the ratio of expression level of a Th1 gene signature and that of a Th2 gene signature can be the ratio of the expression level of TBX21 to the expression level of GATA3. The reference ratio can be the average ratio of the expression levels of the two gene signatures in a reference cell determined by a person of ordinary skill in the art, such as naive CD4+ cells. The reference ratio can also be a cut-off value determined by a person of ordinary skill in the art through statistical analysis.

The expression level of a gene signature can refer to the protein level of the gene signature, or the RNA level of the gene signature. In some embodiments, the expression level of a gene signature refers to the protein level of the gene signature, and methods provided herein include determining the protein level of the gene signature.

In some embodiments, the expression level of a gene signature refers to the mRNA level of the gene signature, and methods provided herein include determining the mRNA level of a gene signature. Methods to determine the mRNA level of a gene in a sample are well known in the art. For example, in some embodiments, the mRNA level can be determined by Polymerase Chain Reaction (PCR), qPCR, qRT-PCR, RNA-seq, microarray analysis, SAGE, MassARRAY technique, next-generation sequencing, or FISH.

Exemplary methods of detecting or quantitating mRNA levels include but are not limited to PCR-based methods, northern blots, ribonuclease protection assays, and the like. The mRNA sequence (e.g., the mRNA of a gene signature, such as CRBN or a CAP, or a fragment thereof) can be used to prepare a probe that is at least partially complementary. The probe can then be used to detect the mRNA sequence in a sample, using any suitable assay, such as PCR-based methods, Northern blotting, a dipstick assay, and the like.

The commonly used methods known in the art for the quantification of mRNA expression in a sample include northern blotting and in situ hybridization (Parker &Barnes, Methods in Molecular Biology 106:247-283 (1999)); RNAse protection assays (Hod, Biotechniques 13:852-854 (1992)); and polymerase chain reaction (PCR) (Weis et ah, Trends in Genetics 8:263-264 (1992)). Alternatively, antibodies may be employed that can recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS).

A sensitive and flexible quantitative method is PCR. Examples of PCR methods can be found in the literature. Examples of PCR assays can be found in U.S. Pat. No. 6,927,024, which is incorporated by reference herein in its entirety. Examples of RT-PCR methods can be found in U.S. Pat. No. 7,122,799, which is incorporated by reference herein in its entirety. A method of fluorescent in situ PCR is described in U.S. Pat. No. 7,186,507, which is incorporated by reference herein in its entirety.

It is noted, however, that other nucleic acid amplification protocols (i.e., other than PCR) may also be used in the nucleic acid analytical methods described herein. For example, suitable amplification methods include ligase chain reaction (see, e.g., Wu & Wallace, Genomics 4:560-569, 1988); strand displacement assay (see, e.g., Walker et al., Proc. Natl. Acad. Sci. USA 89:392-396, 1992; U.S. Pat. No. 5,455,166); and several transcription-based amplification systems, including the methods described in U.S. Pat. Nos. 5,437,990; 5,409,818; and 5,399,491; the transcription amplification system (TAS) (Kwoh et al., Proc. Natl. Acad. Sci. USA 86: 1173-1177, 1989); and self-sustained sequence replication (3SR) (Guatelli et al., Proc. Natl. Acad. Sci. USA 87: 1874-1878, 1990; WO 92/08800). Alternatively, methods that amplify the probe to detectable levels can be used, such as Q-replicase amplification (Kramer & Lizardi, Nature 339:401-402, 1989; Lomeli et al., Clin. Chem. 35: 1826-1831, 1989). A review of known amplification methods is provided, for example, by Abramson and Myers in Current Opinion in Biotechnology 4:41-47 (1993).

mRNA can be isolated from the sample. The sample can be a tissue sample. The tissue sample can be a tumour biopsy, such as a lymph node biopsy. General methods for mRNA extraction are well known in the art and are disclosed in standard textbooks of molecular biology, including Ausubel et al., Current Protocols of Molecular Biology, John Wiley and Sons (1997). In particular, RNA isolation can be performed using purification kit, buffer set and protease from commercial manufacturers, such as Qiagen, according to the manufacturer's instructions. For example, total RNA from cells in culture can be isolated using Qiagen RNeasy mini-columns. Other commercially available RNA isolation kits include MASTERPURE® Complete DNA and RNA Purification Kit (EPICENTRE®, Madison, Wis.), and Paraffin Block RNA Isolation Kit (Ambion, Inc.). Total RNA from tissue samples can be isolated using RNA Stat-60 (Tel-Test). RNA prepared from tumor can be isolated, for example, by cesium chloride density gradient centrifugation.

In some embodiments, the first step in gene expression profiling by PCR is the reverse transcription of the RNA template into cDNA, followed by its exponential amplification in a PCR reaction. In other embodiments, a combined reverse-transcription-polymerase chain reaction (RT-PCR) reaction may be used, e.g., as described in U.S. Pat. Nos. 5,310,652; 5,322,770; 5,561,058; 5,641,864; and 5,693,517. The two commonly used reverse transcriptases are avilo myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GENEAMP™ RNA PCR kit (Perkin Elmer, Calif, USA), following the manufacturer's instructions. The derived cDNA can then be used as a template in the subsequent PCR reaction.

In some embodiments, Real-Time Reverse Transcription-PCR (qRT-PCR) can be used for both the detection and quantification of RNA targets (Bustin, et al., 2005, Clin. Sci., 109:365-379). Examples of qRT-PCR-based methods can be found, for example, in U.S. Pat. No. 7,101,663, which is incorporated by reference herein in its entirety. Instruments for real-time PCR, such as the Applied Biosystems 7500, are available commercially, as are the reagents, such as TaqMan Sequence Detection chemistry.

For example, TaqMan® Gene Expression Assays can be used, following the manufacturer's instructions. These kits are pre-formulated gene expression assays for rapid, reliable detection and quantification of human, mouse and rat mRNA transcripts. TaqMan® or 5′-nuclease assay, as described in U.S. Pat. Nos. 5,210,015; 5,487,972; and 5,804,375; and Holland et al., 1988, Proc. Natl. Acad. Sci. USA 88:7276-7280, can be used. TAQMAN® PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

Any method suitable for detecting degradation product can be used in a 5′ nuclease assay. Often, the detection probe is labeled with two fluorescent dyes, one of which is capable of quenching the fluorescence of the other dye. The dyes are attached to the probe, preferably one attached to the 5′ terminus and the other is attached to an internal site, such that quenching occurs when the probe is in an unhybridized state and such that cleavage of the probe by the 5′ to 3′ exonuclease activity of the DNA polymerase occurs in between the two dyes.

Amplification results in cleavage of the probe between the dyes with a concomitant elimination of quenching and an increase in the fluorescence observable from the initially quenched dye. The accumulation of degradation product is monitored by measuring the increase in reaction fluorescence. U.S. Pat. Nos. 5,491,063 and 5,571,673, both incorporated herein by reference, describe alternative methods for detecting the degradation of probe which occurs concomitant with amplification. 5′-Nuclease assay data may be initially expressed as Ct, or the threshold cycle. As discussed above, fluorescence values are recorded during every cycle and represent the amount of product amplified to that point in the amplification reaction. The point when the fluorescent signal is first recorded as statistically significant is the threshold cycle (Ct).

To minimize errors and the effect of sample-to-sample variation, PCR is usually performed using an internal standard. The ideal internal standard is expressed at a constant level among different tissues, and is unaffected by the experimental treatment. RNAs most frequently used to normalize patterns of gene expression are mRNAs for the housekeeping genes glyceraldehyde-3-phosphate-dehydrogenase (GAPDH) and P-actin.

PCR primers and probes are designed based upon intron sequences present in the gene to be amplified. In this embodiment, the first step in the primer/probe design is the delineation of intron sequences within the genes. This can be done by publicly available software, such as the DNA BLAT software developed by Kent, W., Genome Res. 12(4):656-64 (2002), or by the BLAST software including its variations. Subsequent steps follow well established methods of PCR primer and probe design.

In order to avoid non-specific signals, it can be important to mask repetitive sequences within the introns when designing the primers and probes. This can be easily accomplished by using the Repeat Masker program available on-line through the Baylor College of Medicine, which screens DNA sequences against a library of repetitive elements and returns a query sequence in which the repetitive elements are masked. The masked intron sequences can then be used to design primer and probe sequences using any commercially or otherwise publicly available primer/probe design packages, such as Primer Express (Applied Biosystems); MGB assay-by-design (Applied Biosystems); Primer3 (Rozen and Skaletsky (2000) Primer3 on the WWW for general users and for biologist programmers. In: Krawetz S, Misener S (eds) Bioinformatics Methods and Protocols: Methods in Molecular Biology. Humana Press, Totowa, N.J., pp 365-386).

RNA-Seq, also called Whole Transcriptome Shotgun Sequencing (WTSS) refers to the use of high-throughput sequencing technologies to sequence cDNA in order to get information about a sample's RNA content. Publications describing RNA-Seq include: Wang et al., Nature Reviews Genetics 10 (1): 57-63 (January 2009); Ryan et al. BioTechniques 45 (1): 81-94 (2008); and Maher et al., Nature 458 (7234): 97-101 (January 2009); which are hereby incorporated in their entirety.

Differential gene expression can also be identified, or confirmed using the microarray technique. In this method, polynucleotide sequences of interest (including cDNAs and oligonucleotides) are plated, or arrayed, on a microchip substrate. The arrayed sequences are then hybridized with specific DNA probes from cells or tissues of interest.

In an embodiment of the microarray technique, PCR amplified inserts of cDNA clones are applied to a substrate in a dense array. Preferably at least 10,000 nucleotide sequences are applied to the substrate. The microarrayed genes, immobilized on the microchip at 10,000 elements each, are suitable for hybridization under stringent conditions. Fluorescently labeled cDNA probes may be generated through incorporation of fluorescent nucleotides by reverse transcription of RNA extracted from tissues of interest. Labeled cDNA probes applied to the chip hybridize with specificity to each spot of DNA on the array. After stringent washing to remove non-specifically bound probes, the chip is scanned by confocal laser microscopy or by another detection method, such as a CCD camera. Quantitation of hybridization of each arrayed element allows for assessment of corresponding mRNA abundance. With dual color fluorescence, separately labeled cDNA probes generated from two sources of RNA are hybridized pairwise to the array. The relative abundance of the transcripts from the two sources corresponding to each specified gene is thus determined simultaneously. The miniaturized scale of the hybridization affords a convenient and rapid evaluation of the expression pattern for large numbers of genes. Such methods have been shown to have the sensitivity required to detect rare transcripts, which are expressed at a few copies per cell, and to reproducibly detect at least approximately two-fold differences in the expression levels (Schena et al., Proc. Natl. Acad. Sci. USA 93(2): 106-149 (1996)). Microarray analysis can be performed by commercially available equipment, following manufacturer's protocols, such as by using the Affymetrix GENCHIP™ technology, or Incyte's microarray technology.

Serial analysis of gene expression (SAGE) is a method that allows the simultaneous and quantitative analysis of a large number of gene transcripts, without the need of providing an individual hybridization probe for each transcript. First, a short sequence tag (about 10-14 bp) is generated that contains sufficient information to uniquely identify a transcript, provided that the tag is obtained from a unique position within each transcript. Then, many transcripts are linked together to form long serial molecules, that can be sequenced, revealing the identity of the multiple tags simultaneously. The expression pattern of any population of transcripts can be quantitatively evaluated by determining the abundance of individual tags, and identifying the gene corresponding to each tag. For more details see, e.g. Velculescu et ah, Science 270:484-487 (1995); and Velculescu et al, Cell 88:243-51 (1997).

The MassARRAY (Sequenom, San Diego, Calif.) technology is an automated, high-throughput method of gene expression analysis using mass spectrometry (MS) for detection. According to this method, following the isolation of RNA, reverse transcription and PCR amplification, the cDNAs are subjected to primer extension. The cDNA-derived primer extension products are purified, and dispensed on a chip array that is pre-loaded with the components needed for MALTI-TOF MS sample preparation. The various cDNAs present in the reaction are quantitated by analyzing the peak areas in the mass spectrum obtained.

mRNA level can also be measured by an assay based on hybridization. A typical mRNA assay method can contain the steps of 1) obtaining surface-bound subject probes; 2) hybridization of a population of mRNAs to the surface-bound probes under conditions sufficient to provide for specific binding (3) post-hybridization washes to remove nucleic acids not bound in the hybridization; and (4) detection of the hybridized mRNAs. The reagents used in each of these steps and their conditions for use may vary depending on the particular application.

Any suitable assay platform can be used to determine the mRNA level in a sample. For example, an assay can be in the form of a dipstick, a membrane, a chip, a disk, a test strip, a filter, a microsphere, a slide, a multiwell plate, or an optical fiber. An assay system can have a solid support on which a nucleic acid corresponding to the mRNA is attached. The solid support can have, for example, a plastic, silicon, a metal, a resin, glass, a membrane, a particle, a precipitate, a gel, a polymer, a sheet, a sphere, a polysaccharide, a capillary, a film a plate, or a slide. The assay components can be prepared and packaged together as a kit for detecting an mRNA.

The nucleic acid can be labeled, if desired, to make a population of labeled mRNAs. In general, a sample can be labeled using methods that are well known in the art (e.g., using DNA ligase, terminal transferase, or by labeling the RNA backbone, etc.; see, e.g., Ausubel, et al., Short Protocols in Molecular Biology, 3rd ed., Wiley & Sons 1995 and Sambrook et al., Molecular Cloning: A Laboratory Manual, Third Edition, 2001 Cold Spring Harbor, N.Y.). In some embodiments, the sample is labeled with fluorescent label. Exemplary fluorescent dyes include but are not limited to xanthene dyes, fluorescein dyes, rhodamine dyes, fluorescein isothiocyanate (FITC), 6 carboxyfluorescein (FAM), 6 carboxy-2′,4′,7′,4,7-hexachlorofluorescein (HEX), 6 carboxy 4′, 5′ dichloro 2′, 7′ dimethoxyfluorescein (JOE or J), N,N,N′,N′ tetramethyl 6 carboxyrhodamine (TAMRA or T), 6 carboxy X rhodamine (ROX or R), 5 carboxyrhodamine 6G (R6G5 or G5), 6 carboxyrhodamine 6G (R6G6 or G6), and rhodamine 110; cyanine dyes, e.g. Cy3, Cy5 and Cy7 dyes; Alexa dyes, e.g. Alexa-fluor-555; coumarin, Diethylaminocoumarin, umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, BODIPY dyes, quinoline dyes, Pyrene, Fluorescein Chlorotriazinyl, R110, Eosin, JOE, R6G, Tetramethylrhodamine, Lissamine, ROX, Napthofluorescein, and the like.

Hybridization can be carried out under suitable hybridization conditions, which may vary in stringency as desired. Typical conditions are sufficient to produce probe/target complexes on a solid surface between complementary binding members, i.e., between surface-bound subject probes and complementary mRNAs in a sample. In certain embodiments, stringent hybridization conditions can be employed.

Hybridization is typically performed under stringent hybridization conditions. Standard hybridization techniques (e.g. under conditions sufficient to provide for specific binding of target mRNAs in the sample to the probes) are described in Kallioniemi et al., Science 258:818-821 (1992) and WO 93/18186. Several guides to general techniques are available, e.g., Tijssen, Hybridization with Nucleic Acid Probes, Parts I and II (Elsevier, Amsterdam 1993). For descriptions of techniques suitable for in situ hybridizations, see Gall et al. Meth. Enzymol., 21:470-480 (1981); and Angerer et al. in Genetic Engineering: Principles and Methods (Setlow and Hollaender, Eds.) Vol 7, pgs 43-65 (Plenum Press, New York 1985). Selection of appropriate conditions, including temperature, salt concentration, polynucleotide concentration, hybridization time, stringency of washing conditions, and the like will depend on experimental design, including source of sample, identity of capture agents, degree of complementarity expected, etc., and may be determined as a matter of routine experimentation for those of ordinary skill in the art. Those of ordinary skill will readily recognize that alternative but comparable hybridization and wash conditions can be utilized to provide conditions of similar stringency.

After the mRNA hybridization procedure, the surface bound polynucleotides are typically washed to remove unbound nucleic acids. Washing may be performed using any convenient washing protocol, where the washing conditions are typically stringent, as described above. The hybridization of the target mRNAs to the probes is then detected using standard techniques.

Any methods as described herein or otherwise known in the art can be used to determine the mRNA level of a gene signature in a sample from a subject having MDS. By way of example, in some embodiments, provided herein are methods to treat MDS in a subject that include determining the mRNA level of a gene signature for Th1 cells in a sample from the subject by using qRT-PCR, and administering a therapeutically effective amount of an FTI to the subject if the mRNA level of the gene signature in the sample is higher than a reference expression level of the gene signature. The gene signature for Th1 cells can be selected from the group consisting of TBX21, STAT1, STAT6, CXCR3, CCR5, IFN-γ, TNF-α, IL-2, and IL-12. The sample can be a tissue sample, or a tumor sample. The sample can be a blood sample, or a lymph node sample. The FTI can be tipifarnib. In some embodiments, the gene signature is TBX21 and the methods provided herein include determining the mRNA level of TBX21 in a tumor sample from the subject, and administering a therapeutically effective amount of tipifarnib to the subject if the mRNA level of the TBX21 in the tumor sample is higher than the average expression level of TBX21 in naive CD4+ cells.

In some embodiments, provided herein are methods to treat MDS in a subject that include determining the ratio of the mRNA level of a Th1 gene signature to that of a Th2 gene signature in a sample from the subject by using multiplexing PCR, and administering a therapeutically effective amount of an FTI to the subject if the ratio is higher than a reference ratio. The Th1 gene signature can be selected from the group consisting of TBX21, STAT1, STAT6, CXCR3, CCR5, IFN-γ, TNF-α, IL-2, and IL-12. The Th2 gene signature can be selected from the group consisting of GATA3, CCR4, IL-4, IL-5, IL-6, IL-10, and IL-13. The sample can be a tissue sample, or a tumor sample. The sample can be a blood sample, or a lymph node sample. The FTI can be tipifarnib. In some embodiments, the methods provided herein include determining ratio of the mRNA level of TBX21 to that of GATA3 in a sample from the subject by using multiplexing PCR, and administering a therapeutically effective amount of tipifarnib to the subject if the ratio in the tumor sample is higher than the average ratio of TBX21 expression level to GATA3 expression level in naive CD4+ cells.

Methods to determine a protein level of a gene in a sample are well known in the art. For example, in some embodiments, the protein level can be determined by an immunohistochemistry (IHC) assay, an immunoblotting (TB) assay, an immunofluorescence (IF) assay, flow cytometry (FACS), or an Enzyme-Linked Immunosorbent Assay (ELISA). In some embodiments, the protein level can be determined by Hematoxylin and Eosin stain (“H&E staining”).

The protein level of the gene signature can be detected by a variety of (IHC) approaches or other immunoassay methods. IHC staining of tissue sections has been shown to be a reliable method of assessing or detecting presence of proteins in a sample. Immunohistochemistry techniques utilize an antibody to probe and visualize cellular antigens in situ, generally by chromogenic or fluorescent methods. Thus, antibodies or antisera, including for example, polyclonal antisera, or monoclonal antibodies specific for each gene signature are used to detect expression. As discussed in greater detail below, the antibodies can be detected by direct labelling of the antibodies themselves, for example, with radioactive labels, fluorescent labels, hapten labels such as, biotin, or an enzyme such as horse radish peroxidase or alkaline phosphatase. Alternatively, unlabeled primary antibody is used in conjunction with a labeled secondary antibody, comprising antisera, polyclonal antisera or a monoclonal antibody specific for the primary antibody. Immunohistochemistry protocols and kits are well known in the art and are commercially available. Automated systems for slide preparation and IHC processing are available commercially. The Ventana® BenchMark XT system is an example of such an automated system.

Standard immunological and immunoassay procedures can be found in Basic and Clinical Immunology (Stites & Terr eds., 7th ed. 1991). Moreover, the immunoassays can be performed in any of several configurations, which are reviewed extensively in Enzyme Immunoassay (Maggio, ed., 1980); and Harlow & Lane, supra. For a review of the general immunoassays, see also Methods in Cell Biology: Antibodies in Cell Biology, volume 37 (Asai, ed. 1993); Basic and Clinical Immunology (Stites & Ten, eds., 7th ed. 1991).

Commonly used assays to detect protein level of a gene signature include noncompetitive assays, e.g., sandwich assays, and competitive assays. Typically, an assay such as an ELISA assay can be used. ELISA assays are known in the art, e.g., for assaying a wide variety of tissues and samples, including blood, plasma, serum, a tumor biopsy, a lymph node, or bone marrow.

A wide range of immunoassay techniques using such an assay format are available, see, e.g., U.S. Pat. Nos. 4,016,043, 4,424,279, and 4,018,653, which are hereby incorporated by reference in their entireties. These include both single-site and two-site or “sandwich” assays of the non-competitive types, as well as in the traditional competitive binding assays. These assays also include direct binding of a labeled antibody to a target gene signature. Sandwich assays are commonly used assays. A number of variations of the sandwich assay technique exist. For example, in a typical forward assay, an unlabelled antibody is immobilized on a solid substrate, and the sample to be tested brought into contact with the bound molecule. After a suitable period of incubation, for a period of time sufficient to allow formation of an antibody-antigen complex, a second antibody specific to the antigen, labeled with a reporter molecule capable of producing a detectable signal is then added and incubated, allowing time sufficient for the formation of another complex of antibody-antigen-labeled antibody. Any unreacted material is washed away, and the presence of the antigen is determined by observation of a signal produced by the reporter molecule. The results may either be qualitative, by simple observation of the visible signal, or may be quantitated by comparing with a control sample containing known amounts of the gene signature.

Variations on the forward assay include a simultaneous assay, in which both sample and labeled antibody are added simultaneously to the bound antibody. These techniques are well known to those skilled in the art, including any minor variations as will be readily apparent. In a typical forward sandwich assay, a first antibody having specificity for the gene signature is either covalently or passively bound to a solid surface. The solid surface may be glass or a polymer, the most commonly used polymers being cellulose, polyacrylamide, nylon, polystyrene, polyvinyl chloride, or polypropylene. The solid supports may be in the form of tubes, beads, discs of microplates, or any other surface suitable for conducting an immunoassay. The binding processes are well-known in the art and generally consist of cross-linking covalently binding or physically adsorbing, the polymer-antibody complex is washed in preparation for the test sample. An aliquot of the sample to be tested is then added to the solid phase complex and incubated for a period of time sufficient (e.g. 2-40 minutes or overnight if more convenient) and under suitable conditions (e.g., from room temperature to 40° C. such as between 25° C. and 32° C. inclusive) to allow binding of any subunit present in the antibody. Following the incubation period, the antibody subunit solid phase is washed and dried and incubated with a second antibody specific for a portion of the gene signature. The second antibody is linked to a reporter molecule which is used to indicate the binding of the second antibody to the molecular marker.

In some embodiments, flow cytometry (FACS) can be used to detect the protein level of a gene signature that is expressed on the surface of the cells. Gene signatures that are surface proteins (such as CXCR3) can be detected using antibodies against these gene signatures. The flow cytometer detects and reports the intensity of the fluorichrome-tagged antibody, which indicates the expression level of the gene signature. Non-fluorescent cytoplasmic proteins can also be observed by staining permeabilized cells. The stain can either be a fluorescence compound able to bind to certain molecules, or a fluorichrome-tagged antibody to bind the molecule of choice.

An alternative method involves immobilizing the target gene signature in the sample and then exposing the immobilized target to specific antibody which may or may not be labeled with a reporter molecule. Depending on the amount of target and the strength of the reporter molecule signal, a bound target may be detectable by direct labeling with the antibody. Alternatively, a second labeled antibody, specific to the first antibody is exposed to the target-first antibody complex to form a target-first antibody-second antibody tertiary complex. The complex is detected by the signal emitted by a labeled reporter molecule.

In the case of an enzyme immunoassay, an enzyme is conjugated to the second antibody, generally by means of glutaraldehyde or periodate. As will be readily recognized, however, a wide variety of different conjugation techniques exist, which are readily available to the skilled artisan. Commonly used enzymes include horseradish peroxidase, glucose oxidase, beta-galactosidase, and alkaline phosphatase, and other are discussed herein. The substrates to be used with the specific enzymes are generally chosen for the production, upon hydrolysis by the corresponding enzyme, of a detectable color change. Examples of suitable enzymes include alkaline phosphatase and peroxidase. It is also possible to employ fluorogenic substrates, which yield a fluorescent product rather than the chromogenic substrates noted above. In all cases, the enzyme-labeled antibody is added to the first antibody-molecular marker complex, allowed to bind, and then the excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of gene signature which was present in the sample. Alternately, fluorescent compounds, such as fluorescein and rhodamine, can be chemically coupled to antibodies without altering their binding capacity. When activated by illumination with light of a particular wavelength, the fluorochrome-labeled antibody adsorbs the light energy, inducing a state to excitability in the molecule, followed by emission of the light at a characteristic color visually detectable with a light microscope. As in the EIA, the fluorescent labeled antibody is allowed to bind to the first antibody-molecular marker complex. After washing off the unbound reagent, the remaining tertiary complex is then exposed to the light of the appropriate wavelength, the fluorescence observed indicates the presence of the molecular marker of interest. Immunofluorescence and EIA techniques are both very well established in the art and are discussed herein.

Any methods as described herein or otherwise known in the art can be used to determine the protein level of a gene signature in a sample from a subject having MDS. In some embodiments, the MDS can be lower risk MDS. By way of example, in some embodiments, provided herein are methods to treat MDS in a subject that include determining the protein level of a Th1 gene signature in a sample from the subject by using an IF assay, and administering a therapeutically effective amount of an FTI to the subject if the protein level of the Th1 gene signature in the sample is higher than a reference expression level of the gene signature. The gene signature for Th1 cells can be selected from the group consisting of TBX21, STAT1, STAT6, CXCR3, CCR5, IFN-γ, TNF-α, IL-2, and IL-12. The sample can be a tissue sample, or a tumor sample. The sample can be a blood sample, or a lymph node sample. The FTI can be tipifarnib. In some embodiments, the gene signature is TBX21 and the methods provided herein include determining the protein level of TBX21 in a tumor sample from the subject by an IHC approach, and administering a therapeutically effective amount of tipifarnib to the subject if the protein level of the TBX21 in the tumor sample is higher than a reference expression level of TBX21.

In some embodiments, provided herein are methods to treat MDS in a subject that include determining the ratio of the protein level of a Th1 gene signature to that of a Th2 gene signature in a sample from the subject by using multiplexing ELISA, and administering a therapeutically effective amount of an FTI to the subject if the ratio is higher than a reference ratio. The Th1 gene signature can be selected from the group consisting of TBX21, STAT1, STAT6, CXCR3, CCR5, IFN-γ, TNF-α, IL-2, and IL-12. The Th2 gene signature can be selected from the group consisting of GATA3, CCR4, IL-4, IL-5, IL-6, IL-10, and IL-13. The sample can be a tissue sample, or a tumor sample. The sample can be a blood sample, or a lymph node sample. The FTI can be tipifarnib. In some embodiments, the MDS can be lower risk MDS. In some embodiments, the methods provided herein include determining ratio of the protein level of TBX21 to that of GATA3 in a sample from the subject by using multiplexing ELISA, and administering a therapeutically effective amount of tipifarnib to the subject if the ratio in the tumor sample is higher than the average ratio of TBX21 protein level to GATA3 protein expression level in naive CD4+ cells.

Accordingly, a person of ordinary skill in the art would understand that the methods described herein include using any permutation or combination of the Th1 and Th2 gene signatures and approaches to determine the expression levels of the gene signatures as described herein to identify or select the subjects having MDS and characterized by Th1 dominance. The expression level can be protein level or mRNA level.

In some embodiments, the subject having MDS is determined to be characterized by Th1 dominance by the relatively high number of Th1 cells in a sample from the subject. The sample can be a peripheral blood sample, a lymph node sample, or any other samples disclosed herein. As such, provided herein are methods of selecting MDS patients for an FTI treatment, methods of predicating responsiveness of a subject having MDS to an FTI treatment, methods of increasing the responsiveness to an FTI treatment of a MDS patient population, that include analyzing the cell constitution of a sample from the subject having MDS, or samples from the subject having MDS population. In some embodiments, a subject having MDS is selected for an FTI treatment if the ratio of Th1 cells to Th2 cells in a sample from the subject is higher than a reference ratio. In some embodiments, a subject having MDS is predicted to be likely to respond to an FTI treatment if the ratio of Th1 cells to Th2 cells in a sample from the subject is higher than a reference ratio. In some embodiments, the MDS can be lower risk MDS. In some embodiments, the FTI is tipifarnib.

In some embodiments, provided herein are methods of treating MDS in a subject characterized by Th1 dominance, including analyzing the cell constitution of a sample from a subject having MDS, and administering a therapeutically effective amount of an FTI to the subject if ratio of Th1 cells to Th2 cells in the sample is higher than a reference ratio. The reference ratio can be 1, 2, 3, 4, 5, 6, 7, 8, 9, 10, 20, 30, 40, 50, 75, or 100.

In some embodiments, the ratio of Th1 cells to Th2 cells in a sample from the subject having MDS is at least 2, at least 3, at least 4, at least 5, at least 6, at least 7, at least 8, at least 9, at least 10, at least 20, at least 30, at least 40, at least 50, at least 75, or at least 100. In some embodiments, at least 20% of the cells in a sample from the subject are Th1 cells. In some embodiments, at least 30% of the cells in a sample from the subject are Th1 cells. In some embodiments, at least 40% of the cells in a sample from the subject are Th1 cells. In some embodiments, at least 50% of the cells in a sample from the subject are Th1 cells. In some embodiments, at least 60% of the cells in a sample from the subject are Th1 cells. In some embodiments, at least 70% of the cells in a sample from the subject are Th1 cells. In some embodiments, at least 80% of the cells in a sample from the subject are Th1 cells. In some embodiments, at least 90% of the cells in a sample from the subject are Th1 cells.

Methods to analyze the cell constitution of a sample from a subject are well known in the art, including such as an immunohistochemistry (IHC) assay, an immunofluorescence (IF) assay, and flow cytometry (FACS). Th1 cells differ from other subtypes of helper T cells, such as Th2 cells by, for example, expressing a unique set of cytokines and chemokine receptors on the surface of the cells, which can be used to identify a Th1 cell from a cell population through the approaches as described herein or otherwise known in the art.

In some embodiments, the cell constitution is determined by an IHC assay. A variety of IHC assays are described herein. By way of example, in some embodiments, an IHC staining can be performed on deparaffinised tissue section with anti-TBX21 antibody (staining Th1 cells)(e.g. mouse monoclonal antibody, clone 4B10, 1:100; BD Biosciences) and/or anti-GATA3 (staining Th2 cells) (e.g. mouse monoclonal antibody, clone HG3-35, 1:25; Santa Cruz Biotechnology) overnight at 4° C., after peroxidise and protein blocking. The microwave epitope retrieval in 10 mM Tris/HCl PH9 containing 1 mM ethylenediamine tetraacetic acid can be used for both antibodies, and appropriate negative control (no primary antibody) and positive controls (tonsil or breast tumor sections) can be stained in parallel with each set of tumor studied. See e.g., Iqbal et al., Blood 123(19): 2915-23 (2014). The numbers of Th1 cells in the sample (stained positive for TBX21) and Th2 cells in the sample (stained positive for GATA3) can be determined.

In some embodiments, the cell constitution is determined by flow cytometry (FACS). Various methods of using FACS to identify and enumerate specific T cell subsets are well known in the art and commercially available. The cell surface markers can be used to identify a specific cell population. The Th1 cells can be identified, sorted, and/or enumerated by differential expression of Th1 cell surface markers, for example, CD4 and CXCR3. The Th2 cells can be identified, sorted, and/or enumerated by expression of Th2 cell surface markers, for example, CD4 and CCR4. In some embodiments, the Th1 dominance is determined by enumeration of CD4+ T cell sets. For example, the CD4+ T cells can be divided into four subpopulations based on the presence or absence of the two surface markers: CXCR3 (i.e. CD183) and CCR6 (i.e. CD196). Specifically, Th1 cells (CD183+CD196−); Th1/17 cells (CD183+CD196+); Th2 cells (CD183−CD196−); and Th17 cells (CD183−CD196+). A patient is indicated Th1 dominance if the Th1 cell subpopulation outnumbers the other populations. A patient is indicated Th1/17 dominance if the Th1/17 cell subpopulation outnumbers the other populations. Similarly, a patient is indicated Th2 or Th17 dominance if the Th2 or Th17 cell subpopulation outnumbers the other populations. As Th1/17 cells can be considered a subpopulation of Th1 cells, Th1/17 dominance is considered a subtype of Th1 dominance.

While some cell surface markers are expressed on more than one cell type (e.g. CD4 is expressed on at least both Th1 cells and Th2 cells), flow cytometry staining allow immunophenotyping cells with two or more antibodies simultaneously. By evaluating the unique repertoire of cell surface markers using several antibodies together, each coupled with a different fluorochromes, a given cell population can be identified and quantified. The available technologies include the multicolour flow cytometry technology by BD Biosciences, flow cytometry immunophenotyping technology by Abcam, etc. Various gating and data analysis strategies can be used to distinguish the Th1 cell population.

In some embodiments, provided herein are methods that include analyzing the cell constitution of a blood sample from a subject having MDS using flow cytometry, and administering a therapeutically effective amount of an FTI to the subject if the sample has more Th1 cells than Th2 cells. In some embodiments, the MDS can be lower risk MDS. In some embodiments, the FTI is tipifarnib. In some embodiments, at least 50% of the cells in a sample from the patient are Th1 cells.

As a person of ordinary skill in the art would understand, any methods of T cell profiling known in the art can be used in the methods provided herein to determine the Th1/Th2 balance in a subject, and select the subject for an FTI treatment if the subject is characterized by Th1 dominance. In some embodiments, the T cell constitution of a cell population is determined by the function of the cell population based on cytokine excretion. In some embodiments, a combination of both the surface marker detection and cytokine profiling can be used to determine the Th1/Th2 balance.

Th1 cells and Th2 cells produce different types of cytokines. Cytokines secreted by Th1 cells include, for example, IFN-γ, TNF-α, IL-2, and IL-12, namely, Th1 cytokines. Cytokines secreted by Th2 cells include, for example, IL-4, IL5, and IL-13, namely, Th2 cytokines. Accordingly, the Th1/Th2 balance in a subject can also be determined by analyzing the cytokines in a sample from the subject. In some embodiments, the Th1/Th2 balance in a subject having MDS is characterized by Th1 cytokines in a sample from the subject. The sample can be a whole blood level, a partially purified blood level, a peripheral blood sample, a serum sample, or any other samples disclosed herein. In some embodiments, the sample is a serum sample.

Provided herein are methods of selecting MDS patients for an FTI treatment, methods of predicating responsiveness of a subject having MDS to an FTI treatment, methods of increasing the responsiveness to an FTI treatment of a MDS patient population, that include analyzing the cytokines in a sample from the subject having MDS, or samples from the MDS patient population. In some embodiments, a subject having MDS is selected for an FTI treatment if a sample from the subject has more Th1 cytokines, namely, Th1 cell related cytokines (e.g. IFN-γ, TNF-α, IL-2, or IL-12) than Th2 cytokines, namely, Th2 cell related cytokines (e.g. IL-4, IL5, and IL-13). In some embodiments, a subject having MDS is predicted to be likely responsive to an FTI treatment if a sample from the ratio of Th1 cytokine level to the Th2 cytokine level is higher than a reference ratio. In some embodiments, the MDS can be lower risk MDS. The FTI can be tipifarnib.

In some embodiments, provided herein are methods of treating MDS in a subject characterized by Th1 dominance, that include detecting Th1 cytokine in a sample from the subject, and administering a therapeutically effective amount of an FTI to the subject. The Th1 cytokine can be IFN-γ, TNF-α, IL-2, IL-12, or any combination thereof. In some embodiments, the methods include detecting IFN-γ. In some embodiments, the methods include detecting TNF-α. In some embodiments, the methods include detecting IL-2. In some embodiments, the methods include detecting IL-12. In some embodiments, the methods include detecting at least two Th1 cell related cytokines. In some embodiments, the methods include detecting IFN-γ and TNF-α. In some embodiments, the methods can include detecting at least three Th1 cell related cytokines. In some embodiments, the methods can include detecting at least four Th1 cell related cytokines.

The methods provided herein can further include determining the level of the Th1 cytokine in a sample from a subject having MDS. In some embodiments, provided herein are methods of selecting MDS patients for an FTI treatment, methods of predicating responsiveness of a subject having MDS to an FTI treatment, methods of increasing the responsiveness to an FTI treatment of a MDS patient population, that include determining the level of a Th1 cytokine in a sample from a subject having MDS, or samples from the MDS patient population, wherein a subject is predicted to be likely responsive to the FTI treatment, or a patient is administered with a therapeutically effective amount of a FTI, if the level of a Th1 cytokine in a sample from the patient is higher than a reference level. In some embodiments, the MDS can be lower risk MDS. The FTI can be tipifarnib.

In some embodiment, the methods include determining in a sample from a subject having MDS the level of a Th1 cytokine, and administering a therapeutically effective amount of an FTI to a subject if the level of the Th1 cytokine is higher than a reference level. The Th1 cytokine is selected from the group consisting of IFN-γ, TNF-α, IL-2, and IL-12. The methods can include determining the level of at least one, at least two, at least three, or at least four Th1 cytokines. In some embodiments, the methods can include determining the IFN-γ level. In some embodiments, the methods can include determining the TNF-α level. In some embodiments, the methods can include determining the IFN-γ level and the TNF-α level.

In some embodiments, the methods include determining in a sample from a subject having MDS the IFN-γ level, and administering a therapeutically effective amount of an FTI to a subject if the IFN-γ level is higher than a reference level.

In some embodiments, the methods provided herein further include analyzing a Th2 cytokine in a sample from a subject having MDS, wherein the subject is determined to be likely responsive to an FTI treatment if the Th2 cytokine is absent from the sample, or wherein the subject is not recommended for an FTI treatment if the Th2 cytokine is detected in the sample. In some embodiments, the methods of treating a MDS in a subject include determining the absence of a Th2 cytokine in a sample from the subject, or determining the level of the Th2 cytokine in a sample from MDS subject to be lower than a reference level, and administering a therapeutically effective amount of an FTI to the MDS subject. The Th2 cytokine can include IL-4, IL5, IL-13, or any combination thereof. The FTI can be tipifarnib.

In some embodiments, the methods provided herein further include determining the ratio of the level of a Th1 cytokine to that of a Th2 cytokine in a sample from a subject having MDS, and administering a therapeutically effective amount of an FTI if the ratio is higher than a reference ratio. In some embodiments, the reference ratio can be 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 200, 300, 400, 500, 600, 700, 800, 900, or 1000. In some embodiments, the reference ratio can be 1, 2, 3, 4, 5, 6, 7, 8, 9, 10, 20, 30, 40, 50, 75, or 100. The Th1 cytokine can be IFN-γ, TNF-α, IL-2, or IL-12, and the Th2 cytokine can be IL-4, IL5, IL-13. In some embodiments, the methods provided herein include determining the ratio of the IFN-γ level to the IL-4 level in a sample from a subject having MDS, and administering a therapeutically effective amount of an FTI to the subject if the ratio is higher than a reference ratio. In some embodiments, the reference ratio can be 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 200, 300, 400, 500, 600, 700, 800, 900, or 1000. In some embodiments, the reference ratio can be 1, 2, 3, 4, 5, 6, 7, 8, 9, 10, 20, 30, 40, 50, 75, or 100. In some embodiments, the ratio is at least 50. In some embodiments, the MDS can be lower risk MDS. In some embodiments, the FTI is tipifarnib.

Many approaches are known in the art to detect and/or quantify a cytokine in a sample from a subject, and any available approaches can be used in the methods provided herein to determine the Th1/Th2 balance in a subject. Th1 cytokines are also gene signatures for Th1 cell; and Th2 cytokines are also gene signatures for Th2 cells. Thus, any methods for analyzing expression levels (e.g., the protein level or the mRNA level) of a gene signature as described herein or otherwise known in the art can be used to determine the level of a cytokine in a sample, such as an IHC assay, an IB assay, an IF assay, FACS, ELISA, protein microarray analysis, qPCR, qRT-PCR, RNA-seq, RNA microarray analysis, SAGE, MassARRAY technique, next-generation sequencing, or FISH. In some embodiments, the methods provided herein include analyzing a cytokine in a sample by RT-PCR, microarray, FACS, ELISA, Cytometric Bead Array (“CBA”), or Intracellular cytokine staining (ICS).

A number of kits and/or technology platforms for cytokine profiling or cytokine analysis are also commercially available. For example, QIAGEN provides advanced QPCR technology for cytokines profiling, as well as ELISArray Cytokine kits for simultaneously detect multiple cytokines; BD Biosciences provide ELISA, CBA and ICS related technologies; Full Moon BioSystems provides Cytokine Profiling Antibody Array that provides high-throughput ELISA based antibody array for protein expression profiling of cytokines. Any available approaches can be used in the methods provided herein.

In some embodiments, provided herein are methods that include determining in a sample from a subject having MDS the level of IFN-γ to be higher than a reference level by ICS, and administering a therapeutically effective amount of an FTI to the subject. In some embodiments, the methods also include determining the absence of IL-4 in the sample. In some embodiments, the sample is a serum sample. In some embodiments, the FTI is tipifarnib. As a person of ordinary skill in the art would understand, any methods of cytokine profiling known in the art can be used in the methods provided herein to determine Th1/Th2 balance in a subject, and select the subject for an FTI treatment if the subject is determined to be characterized by Th1 dominance.

Accordingly, a person of ordinary skill in the art would understand that the methods described herein include using any permutation or combination of the Th1 cytokine and Th2 cytokine to identify or select the subjects characterized by Th1 dominance for MDS treatment with FTI and using any permutation or combination approaches to determine the levels of these cytokines as described herein or otherwise known in the art.

The Th1 dominance can also be characterized by the expression of a subset of genes associated with a cytotoxic profile, such as GNLY, PRF, GRMK, GZMH, GZMM, LYZ, CD8β, and KIR molecules including, for example, KIR2DS2, KIR2DS5, KIR3DL1 and KIR3DL2. Thus, methods provided herein can also include determining the expression level of an additional gene signature selected from the group consisting of GNLY, PRF, GRMK, GZMH, GZMM, LYZ, CD8β, KIR2DS2, KIR2DS5, KIR3DL1 and KIR3DL2. In some embodiments, the methods include determining the expression levels of at least two additional gene signatures. In some embodiments, the methods include determining the expression levels of at least three additional gene signatures. In some embodiments, the methods include determining the expression levels of at least four additional gene signatures. In some embodiments, the methods include determining the expression levels of at least five additional gene signatures. In some embodiments, the methods include determining the expression levels of at least six, seven, eight, nine, ten or eleven additional gene signatures. The additional gene signatures can include any of combinations of GNLY, PRF, GRMK, GZMH, GZMM, LYZ, CD8β, KIR2DS2, KIR2DS5, KIR3DL1 and KIR3DL2. In some embodiments, the additional gene signature includes KIR2DS2. In some embodiments, the additional gene signature includes KIR2DS5. In some embodiments, the additional gene signature includes GZMM. In some embodiments, the additional gene signature includes KIR2DS2, KIR2DS5 and GZMM.

In some embodiments, the sample can be a tumor biopsy, a blood sample, a lymph node sample, or any other sample disclosed herein. In some embodiments, the FTI is tipifarnib.

In some embodiments, the methods provided herein further include determining the expression level of an additional gene signature selected from the group consisting of GNLY, PRF, GRMK, GZMH, GZMM, LYZ, CD8β, KIR2DS2, KIR2DS5, KIR3DL1 and KIR3DL2, in a sample from a subject having MDS, and administering a therapeutically effective amount of an FTI to the subject if the level of the additional gene signature in the sample is higher than a reference level. The reference expression level of the additional gene signature can be determined by a person of ordinary skill in the art. In some embodiments, the reference expression level of the additional gene signature is the average expression level of the additional gene signature in naive CD4+ T cells. The expression level of the additional gene signature can be the protein level of the gene signature. The expression level of the additional gene signature can be the mRNA level of the gene signature. In some embodiments, the MDS can be lower risk MDS. In some embodiments, the FTI is tipifarnib.

The ratio of expression levels of certain gene signatures in a sample from a subject having MDS, such as the ratio of the expression level of KIR2DS2 to that of KIR2DL2, or the ratio of the expression level of KIR2DS5 to that of KIR2DL5, can also indicate whether the subject is likely to respond to an FTI treatment. In some embodiments, the methods provided herein further include determining ratio of the expression level of KIR2DS2 to the expression level of KIR2DL2 (the “KIR2DS2/KIR2DL2 ratio”) in a sample from subject having MDS, or determining ratio of the expression level of KIR2DS5 to the expression level of KIR2DL5 (the “KIR2DS5/KIR2DL5 ratio”) in a sample from subject having MDS. In some embodiments, the methods provided herein further include determining KIR2DS2/KIR2DL2 ratio or the KIR2DS5/KIR2DL5 ratio in a sample from a subject having MDS, and administering a therapeutically effective amount of an FTI to the subject if the KIR2DS2/KIR2DL2 ratio or the KIR2DS5/KIR2DL5 ratio is higher than a reference ratio. In some embodiments, the reference ratio can be 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, 200, 300, 400, 500, 600, 700, 800, 900, or 1000. In some embodiments, the MDS can be lower risk MDS. In some embodiments, the FTI can be tipifarnib.

Any methods for analyzing expression levels (e.g., the protein level or the mRNA level) as described herein or otherwise known in the art can be used to determine the level of the additional gene signature in a sample, such as an IHC assay, an IB assay, an IF assay, FACS, ELISA, protein microarray analysis, qPCR, qRT-PCR, RNA-seq, RNA microarray analysis, SAGE, MassARRAY technique, next-generation sequencing, or FISH.

RASGRP (Ras RAS guanyl-releasing protein) are guanine nucleotide exchange factors for HRAS and NRAS. RASGRP can refer to RASGRP1, RASGRP2, RASGRP23, RASGRP4, or any combination thereof. The expression level of RASGRP, such as RASGRP1, in a sample from a subject having MDS can also indicate whether the subject is likely to response to an FTI treatment. Accordingly, in some embodiments, the methods provided herein further include determining the expression level of a RASGRP in a sample from a subject having MDS, and administering a therapeutically effective amount of an FTI to the subject if the expression level of the RASGRP in the sample is higher than a reference expression level of RASGRP. The expression level of the RASGRP can be the protein level of the RASGRP. The expression level of the RASGRP can be the mRNA level of the RASGRP. In some embodiments, the RASGRP refer to RASGRP1, and the methods provided herein further include determining the expression level of a RASGRP1 in a sample from a subject having MDS, and administering a therapeutically effective amount of an FTI to the subject if the expression level of the RASGRP1 in the sample is higher than a reference expression level of RASGRP1.

In some embodiments, the sample can be a tumor biopsy, a blood sample, a lymph node sample, or any other sample disclosed herein. In some embodiments, the FTI is tipifarnib.

Any methods for analyzing expression levels (e.g., the protein level or the mRNA level) as described herein or otherwise known in the art can be used to determine the level of the RASGRP in a sample, such as an IHC assay, an IB assay, an IF assay, FACS, ELISA, protein microarray analysis, qPCR, qRT-PCR, RNA-seq, RNA microarray analysis, SAGE, MassARRAY technique, next-generation sequencing, or FISH.

The mutation status of RhoA (Ras Homolog Family Member A) can also indicate whether a subject having MDS is likely to response to an FTI treatment. Accordingly, in some embodiments, the methods provided herein further include determining the mutation status of RhoA in a sample from a subject having MDS, and administering a therapeutically effective amount of an FTI to the subject if the sample does not have a RhoA mutation. In some embodiments, the sample can be a tumor biopsy, a blood sample, a lymph node sample, or any other sample disclosed herein. In some embodiments, the MDS can be lower risk MDS. In some embodiments, the FTI is tipifarnib.

A variety of approaches are known in the art to determine the mutation status of RhoA in a sample. In some embodiments, the RhoA mutation status is determined by analyzing proteins obtained from a sample. Available approaches include such as an IHC assay, an IB assay, an IF assay, FACS, or ELISA. In some embodiments, the RhoA mutation status is determined at nucleic acid level. Available approaches include such as sequencing, Polymerase Chain Reaction (PCR), DNA microarray, Mass Spectrometry (MS), Single Nucleotide Polymorphism (SNP) assay, denaturing high-performance liquid chromatography (DHPLC), or Restriction Fragment Length Polymorphism (RFLP) assay. In some embodiments, the RhoA mutation status is determined by multiplexing PCR. In some embodiments, the RhoA mutation status is determined by next generation sequencing

Accordingly, a person of ordinary skill in the art would understand that the methods described herein include using any permutation or combination of the additional factors to identify or select the subjects having MDS that are likely responsive to an FTI treatment and using any permutation or combination approaches to determine the presence, absence or levels of these additional markers as described herein or otherwise known in the art.

In some embodiments, provided herein is a method of treating MDS in a subject characterized with Th1 dominance with an FTI or a pharmaceutical composition having FTI. The pharmaceutical compositions provided herein contain therapeutically effective amounts of an FTI and a pharmaceutically acceptable carrier, diluent or excipient. In some embodiments, the FTI is tipifarnib; arglabin; perrilyl alcohol; SCH-66336; L778123; L739749; FTI-277; L744832; R208176; BMS 214662; AZD3409; or CP-609,754. In some embodiments, the MDS can be lower risk MDS. In some embodiments, the FTI is tipifarnib.

The FTI can be formulated into suitable pharmaceutical preparations such as solutions, suspensions, tablets, dispersible tablets, pills, capsules, powders, sustained release formulations or elixirs, for oral administration or in sterile solutions or suspensions for ophthalmic or parenteral administration, as well as transdermal patch preparation and dry powder inhalers. Typically the FTI is formulated into pharmaceutical compositions using techniques and procedures well known in the art (see, e.g., Ansel Introduction to Pharmaceutical Dosage Forms, Seventh Edition 1999).

In the compositions, effective concentrations of the FTI and pharmaceutically acceptable salts is (are) mixed with a suitable pharmaceutical carrier or vehicle. In certain embodiments, the concentrations of the FTI in the compositions are effective for delivery of an amount, upon administration, that treats, prevents, or ameliorates one or more of the symptoms and/or progression of cancer, including haematological cancers and solid tumors.

The compositions can be formulated for single dosage administration. To formulate a composition, the weight fraction of the FTI is dissolved, suspended, dispersed or otherwise mixed in a selected vehicle at an effective concentration such that the treated condition is relieved or ameliorated. Pharmaceutical carriers or vehicles suitable for administration of the FTI provided herein include any such carriers known to those skilled in the art to be suitable for the particular mode of administration.

In addition, the FTI can be formulated as the sole pharmaceutically active ingredient in the composition or may be combined with other active ingredients. Liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as known in the art. Briefly, liposomes such as multilamellar vesicles (MLV's) may be formed by drying down egg phosphatidyl choline and brain phosphatidyl serine (7:3 molar ratio) on the inside of a flask. A solution of an FTI provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

The FTI is included in the pharmaceutically acceptable carrier in an amount sufficient to exert a therapeutically useful effect in the absence of undesirable side effects on the patient treated. The therapeutically effective concentration may be determined empirically by testing the compounds in in vitro and in vivo systems described herein and then extrapolated therefrom for dosages for humans.

The concentration of FTI in the pharmaceutical composition will depend on absorption, tissue distribution, inactivation and excretion rates of the FTI, the physicochemical characteristics of the FTI, the dosage schedule, and amount administered as well as other factors known to those of skill in the art. For example, the amount that is delivered is sufficient to ameliorate one or more of the symptoms of cancer, including hematopoietic cancers and solid tumors.

In certain embodiments, a therapeutically effective dosage should produce a serum concentration of active ingredient of from about 0.1 ng/ml to about 50-100 μg/ml. In one embodiment, the pharmaceutical compositions provide a dosage of from about 0.001 mg to about 2000 mg of compound per kilogram of body weight per day. Pharmaceutical dosage unit forms are prepared to provide from about 1 mg to about 1000 mg and in certain embodiments, from about 10 to about 500 mg of the essential active ingredient or a combination of essential ingredients per dosage unit form.

The FTI may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

Thus, effective concentrations or amounts of one or more of the compounds described herein or pharmaceutically acceptable salts thereof are mixed with a suitable pharmaceutical carrier or vehicle for systemic, topical or local administration to form pharmaceutical compositions. Compounds are included in an amount effective for ameliorating one or more symptoms of, or for treating, retarding progression, or preventing. The concentration of active compound in the composition will depend on absorption, tissue distribution, inactivation, excretion rates of the active compound, the dosage schedule, amount administered, particular formulation as well as other factors known to those of skill in the art.

The compositions are intended to be administered by a suitable route, including but not limited to orally, parenterally, rectally, topically and locally. For oral administration, capsules and tablets can be formulated. The compositions are in liquid, semi-liquid or solid form and are formulated in a manner suitable for each route of administration.

Solutions or suspensions used for parenteral, intradermal, subcutaneous, or topical application can include any of the following components: a sterile diluent, such as water for injection, saline solution, fixed oil, polyethylene glycol, glycerine, propylene glycol, dimethyl acetamide or other synthetic solvent; antimicrobial agents, such as benzyl alcohol and methyl parabens; antioxidants, such as ascorbic acid and sodium bisulfate; chelating agents, such as ethylenediaminetetraacetic acid (EDTA); buffers, such as acetates, citrates and phosphates; and agents for the adjustment of tonicity such as sodium chloride or dextrose. Parenteral preparations can be enclosed in ampules, pens, disposable syringes or single or multiple dose vials made of glass, plastic or other suitable material.

In instances in which the FTI exhibits insufficient solubility, methods for solubilizing compounds can be used. Such methods are known to those of skill in this art, and include, but are not limited to, using cosolvents, such as dimethylsulfoxide (DMSO), using surfactants, such as TWEEN®, or dissolution in aqueous sodium bicarbonate.

Upon mixing or addition of the compound(s), the resulting mixture may be a solution, suspension, emulsion or the like. The form of the resulting mixture depends upon a number of factors, including the intended mode of administration and the solubility of the compound in the selected carrier or vehicle. The effective concentration is sufficient for ameliorating the symptoms of the disease, disorder or condition treated and may be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil water emulsions containing suitable quantities of the compounds or pharmaceutically acceptable salts thereof. The pharmaceutically therapeutically active compounds and salts thereof are formulated and administered in unit dosage forms or multiple dosage forms. Unit dose forms as used herein refer to physically discrete units suitable for human and animal subjects and packaged individually as is known in the art. Each unit dose contains a predetermined quantity of the therapeutically active compound sufficient to produce the desired therapeutic effect, in association with the required pharmaceutical carrier, vehicle or diluent. Examples of unit dose forms include ampules and syringes and individually packaged tablets or capsules. Unit dose forms may be administered in fractions or multiples thereof. A multiple dose form is a plurality of identical unit dosage forms packaged in a single container to be administered in segregated unit dose form. Examples of multiple dose forms include vials, bottles of tablets or capsules or bottles of pints or gallons. Hence, multiple dose form is a multiple of unit doses which are not segregated in packaging.

Sustained-release preparations can also be prepared. Suitable examples of sustained-release preparations include semipermeable matrices of solid hydrophobic polymers containing the compound provided herein, which matrices are in the form of shaped articles, e.g., films, or microcapsule. Examples of sustained-release matrices include iontophoresis patches, polyesters, hydrogels (for example, poly(2-hydroxyethyl-methacrylate), or poly(vinylalcohol)), polylactides, copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated compound remain in the body for a long time, they may denature or aggregate as a result of exposure to moisture at 37° C., resulting in a loss of biological activity and possible changes in their structure. Rational strategies can be devised for stabilization depending on the mechanism of action involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

Dosage forms or compositions containing active ingredient in the range of 0.005% to 100% with the balance made up from non toxic carrier may be prepared. For oral administration, a pharmaceutically acceptable non toxic composition is formed by the incorporation of any of the normally employed excipients, such as, for example pharmaceutical grades of mannitol, lactose, starch, magnesium stearate, talcum, cellulose derivatives, sodium crosscarmellose, glucose, sucrose, magnesium carbonate or sodium saccharin. Such compositions include solutions, suspensions, tablets, capsules, powders and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparation of these compositions are known to those skilled in the art. The contemplated compositions may contain about 0.001% 100% active ingredient, in certain embodiments, about 0.1-85% or about 75-95%.

The FTI or pharmaceutically acceptable salts can be prepared with carriers that protect the compound against rapid elimination from the body, such as time release formulations or coatings.

The compositions can include other active compounds to obtain desired combinations of properties. The compounds provided herein, or pharmaceutically acceptable salts thereof as described herein, can also be administered together with another pharmacological agent known in the general art to be of value in treating one or more of the diseases or medical conditions referred to hereinabove, such as diseases related to oxidative stress.

Lactose-free compositions provided herein can contain excipients that are well known in the art and are listed, for example, in the U.S. Pharmocopia (USP) SP (XXI)/NF (XVI). In general, lactose-free compositions contain an active ingredient, a binder/filler, and a lubricant in pharmaceutically compatible and pharmaceutically acceptable amounts. Exemplary lactose-free dosage forms contain an active ingredient, microcrystalline cellulose, pre-gelatinized starch and magnesium stearate.

Further encompassed are anhydrous pharmaceutical compositions and dosage forms containing a compound provided herein. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment and use of formulations.

Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine are anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are packaged using materials known to prevent exposure to water such that they can be included in suitable formulary kits. Examples of suitable packaging include, but are not limited to, hermetically sealed foils, plastics, unit dose containers (e.g., vials), blister packs and strip packs.

Oral pharmaceutical dosage forms are either solid, gel or liquid. The solid dosage forms are tablets, capsules, granules, and bulk powders. Types of oral tablets include compressed, chewable lozenges and tablets which may be enteric coated, sugar coated or film coated. Capsules may be hard or soft gelatin capsules, while granules and powders may be provided in non effervescent or effervescent form with the combination of other ingredients known to those skilled in the art.

In certain embodiments, the formulations are solid dosage forms, such as capsules or tablets. The tablets, pills, capsules, troches and the like can contain any of the following ingredients, or compounds of a similar nature: a binder; a diluent; a disintegrating agent; a lubricant; a glidant; a sweetening agent; and a flavoring agent.

Examples of binders include microcrystalline cellulose, gum tragacanth, glucose solution, acacia mucilage, gelatin solution, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

Pharmaceutically acceptable carriers included in tablets are binders, lubricants, diluents, disintegrating agents, coloring agents, flavoring agents, and wetting agents. Enteric coated tablets, because of the enteric coating, resist the action of stomach acid and dissolve or disintegrate in the neutral or alkaline intestines. Sugar coated tablets are compressed tablets to which different layers of pharmaceutically acceptable substances are applied. Film coated tablets are compressed tablets which have been coated with a polymer or other suitable coating. Multiple compressed tablets are compressed tablets made by more than one compression cycle utilizing the pharmaceutically acceptable substances previously mentioned. Coloring agents may also be used in the above dosage forms. Flavoring and sweetening agents are used in compressed tablets, sugar coated, multiple compressed and chewable tablets. Flavoring and sweetening agents are especially useful in the formation of chewable tablets and lozenges.

Liquid oral dosage forms include aqueous solutions, emulsions, suspensions, solutions and/or suspensions reconstituted from non effervescent granules and effervescent preparations reconstituted from effervescent granules. Aqueous solutions include, for example, elixirs and syrups. Emulsions are either oil in-water or water in oil.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two phase system in which one liquid is dispersed in the form of small globules throughout another liquid. Pharmaceutically acceptable carriers used in emulsions are non aqueous liquids, emulsifying agents and preservatives. Suspensions use pharmaceutically acceptable suspending agents and preservatives. Pharmaceutically acceptable substances used in non effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic add, sodium benzoate and alcohol. Examples of non aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Diluents include lactose and sucrose. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic adds include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example propylene carbonate, vegetable oils or triglycerides, is encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include, but are not limited to, those containing a compound provided herein, a dialkylated mono- or poly-alkylene glycol, including, but not limited to, 1,2-dimethoxymethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl) acetals of lower alkyl aldehydes such as acetaldehyde diethyl acetal.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

Parenteral administration, generally characterized by injection, either subcutaneously, intramuscularly or intravenously is also provided herein. Injectables can be prepared in conventional forms, either as liquid solutions or suspensions, solid forms suitable for solution or suspension in liquid prior to injection, or as emulsions. Suitable excipients are, for example, water, saline, dextrose, glycerol or ethanol. In addition, if desired, the pharmaceutical compositions to be administered may also contain minor amounts of non toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. Implantation of a slow release or sustained release system, such that a constant level of dosage is maintained is also contemplated herein. Briefly, a compound provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of active compound contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple dose containers which include phenols or cresols, mercurials, benzyl alcohol, chlorobutanol, methyl and propyl p hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcellulose, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles and sodium hydroxide, hydrochloric acid, citric acid or lactic acid for pH adjustment.

The concentration of the FTI is adjusted so that an injection provides an effective amount to produce the desired pharmacological effect. The exact dose depends on the age, weight and condition of the patient or animal as is known in the art. The unit dose parenteral preparations are packaged in an ampule, a vial or a syringe with a needle. All preparations for parenteral administration must be sterile, as is known and practiced in the art.

Illustratively, intravenous or intraarterial infusion of a sterile aqueous solution containing an FTI is an effective mode of administration. Another embodiment is a sterile aqueous or oily solution or suspension containing an active material injected as necessary to produce the desired pharmacological effect.

Injectables are designed for local and systemic administration. Typically a therapeutically effective dosage is formulated to contain a concentration of at least about 0.1% w/w up to about 90% w/w or more, such as more than 1% w/w of the active compound to the treated tissue(s). The active ingredient may be administered at once, or may be divided into a number of smaller doses to be administered at intervals of time. It is understood that the precise dosage and duration of treatment is a function of the tissue being treated and may be determined empirically using known testing protocols or by extrapolation from in vivo or in vitro test data. It is to be noted that concentrations and dosage values may also vary with the age of the individual treated. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope or practice of the claimed formulations.

The FTI can be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.

Of interest herein are also lyophilized powders, which can be reconstituted for administration as solutions, emulsions and other mixtures. They can also be reconstituted and formulated as solids or gels.

The sterile, lyophilized powder is prepared by dissolving an FTI provided herein, or a pharmaceutically acceptable salt thereof, in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides the desired formulation. Generally, the resulting solution will be apportioned into vials for lyophilization. Each vial will contain a single dosage (including but not limited to 10-1000 mg or 100-500 mg) or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, about 1-50 mg, about 5-35 mg, or about 9-30 mg of lyophilized powder, is added per mL of sterile water or other suitable carrier. The precise amount depends upon the selected compound. Such amount can be empirically determined.

Topical mixtures are prepared as described for the local and systemic administration. The resulting mixture may be a solution, suspension, emulsion or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The FTI or pharmaceutical composition having an FTI can be formulated as aerosols for topical application, such as by inhalation (see, e.g., U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will have diameters of less than 50 microns or less than 10 microns.

The FTI or pharmaceutical composition having an FTI can be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered. These solutions, particularly those intended for ophthalmic use, may be formulated as 0.01%-10% isotonic solutions, pH about 5-7, with appropriate salts.

Other routes of administration, such as transdermal patches, and rectal administration are also contemplated herein. For example, pharmaceutical dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin gelatin, carbowax (polyoxyethylene glycol) and appropriate mixtures of mono, di and triglycerides of fatty acids. Combinations of the various bases may be used. Agents to raise the melting point of suppositories include spermaceti and wax. Rectal suppositories may be prepared either by the compressed method or by molding. An exemplary weight of a rectal suppository is about 2 to 3 grams. Tablets and capsules for rectal administration are manufactured using the same pharmaceutically acceptable substance and by the same methods as for formulations for oral administration.

The FTI or pharmaceutical composition having an FTI provided herein can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and U.S. Pat. Nos. 4,008,719, 5,674,533, 5,059,595, 5,591,767, 5,120,548, 5,073,543, 5,639,476, 5,354,556, 5,639,480, 5,733,566, 5,739,108, 5,891,474, 5,922,356, 5,972,891, 5,980,945, 5,993,855, 6,045,830, 6,087,324, 6,113,943, 6,197,350, 6,248,363, 6,264,970, 6,267,981, 6,376,461, 6,419,961, 6,589,548, 6,613,358, 6,699,500 and 6,740,634, each of which is incorporated herein by reference. Such dosage forms can be used to provide slow or controlled-release of FTI using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled-release formulations known to those of ordinary skill in the art, including those described herein, can be readily selected for use with the active ingredients provided herein.

All controlled-release pharmaceutical products have a common goal of improving drug therapy over that achieved by their non-controlled counterparts. In one embodiment, the use of an optimally designed controlled-release preparation in medical treatment is characterized by a minimum of drug substance being employed to cure or control the condition in a minimum amount of time. In certain embodiments, advantages of controlled-release formulations include extended activity of the drug, reduced dosage frequency, and increased patient compliance. In addition, controlled-release formulations can be used to affect the time of onset of action or other characteristics, such as blood levels of the drug, and can thus affect the occurrence of side (e.g., adverse) effects.

Most controlled-release formulations are designed to initially release an amount of drug (active ingredient) that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water, or other physiological conditions or compounds.

In certain embodiments, the FTI can be administered using intravenous infusion, an implantable osmotic pump, a transdermal patch, liposomes, or other modes of administration. In one embodiment, a pump may be used (see, Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials can be used. In yet another embodiment, a controlled release system can be placed in proximity of the therapeutic target, i.e., thus requiring only a fraction of the systemic dose (see, e.g., Goodson, Medical Applications of Controlled Release, vol. 2, pp. 115-138 (1984).

In some embodiments, a controlled release device is introduced into a subject in proximity of the site of inappropriate immune activation or a tumor. Other controlled release systems are discussed in the review by Langer (Science 249:1527-1533 (1990). The F can be dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The active ingredient then diffuses through the outer polymeric membrane in a release rate controlling step. The percentage of active ingredient contained in such parenteral compositions is highly dependent on the specific nature thereof, as well as the needs of the subject.

The FTI or pharmaceutical composition of FTI can be packaged as articles of manufacture containing packaging material, a compound or pharmaceutically acceptable salt thereof provided herein, which is used for treatment, prevention or amelioration of one or more symptoms or progression of cancer, including haematological cancers and solid tumors, and a label that indicates that the compound or pharmaceutically acceptable salt thereof is used for treatment, prevention or amelioration of one or more symptoms or progression of cancer, including haematological cancers and solid tumors.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, pens, bottles, and any packaging material suitable for a selected formulation and intended mode of administration and treatment. A wide array of formulations of the compounds and compositions provided herein are contemplated.

In some embodiments, a therapeutically effective amount of the pharmaceutical composition having an FTI is administered orally or parenterally. In some embodiments, the pharmaceutical composition having tipifarnib as the active ingredient and is administered orally in an amount of from 1 up to 1500 mg/kg daily, either as a single dose or subdivided into more than one dose, or more particularly in an amount of from 10 to 1200 mg/kg daily. In some embodiments, the pharmaceutical composition having tipifarnib as the active ingredient and is administered orally in an amount of 100 mg/kg daily, 200 mg/kg daily, 300 mg/kg daily, 400 mg/kg daily, 500 mg/kg daily, 600 mg/kg daily, 700 mg/kg daily, 800 mg/kg daily, 900 mg/kg daily, 1000 mg/kg daily, 1100 mg/kg daily, or 1200 mg/kg daily. In some embodiments, the FTI is tipifarnib.

In some embodiments, the FTI is administered at a dose of 200-1500 mg daily. In some embodiments, the FTI is administered at a dose of 200-1200 mg daily. In some embodiments, the FTI is administered at a dose of 200 mg daily. In some embodiments, the FTI is administered at a dose of 300 mg daily. In some embodiments, the FTI is administered at a dose of 400 mg daily. In some embodiments, the FTI is administered at a dose of 500 mg daily. In some embodiments, the FTI is administered at a dose of 600 mg daily. In some embodiments, the FTI is administered at a dose of 700 mg daily. In some embodiments, the FTI is administered at a dose of 800 mg daily. In some embodiments, the FTI is administered at a dose of 900 mg daily. In some embodiments, the FTI is administered at a dose of 1000 mg daily. In some embodiments, the FTI is administered at a dose of 1100 mg daily. In some embodiments, the FTI is administered at a dose of 1200 mg daily. In some embodiments, the FTI is administered at a dose of 1300 mg daily. In some embodiments, the FTI is administered at a dose of 1400 mg daily. In some embodiments, the FTI is tipifarnib.

In some embodiments, the FTI is administered at a dose of 200-1400 mg b.i.d. In some embodiments, the FTI is administered at a dose of 300-1200 mg b.i.d. In some embodiments, the FTI is administered at a dose of 300-900 mg b.i.d. In some embodiments, the FTI is administered at a dose of 600 mg b.i.d. In some embodiments, the FTI is administered at a dose of 700 mg b.i.d. In some embodiments, the FTI is administered at a dose of 800 mg b.i.d. In some embodiments, the FTI is administered at a dose of 900 mg b.i.d. In some embodiments, the FTI is administered at a dose of 1000 mg b.i.d. In some embodiments, the FTI is administered at a dose of 1100 mg b.i.d. In some embodiments, the FTI is administered at a dose of 1200 mg b.i.d. In some embodiments, the FTI is tipifarnib.

As a person of ordinary skill in the art would understand, the dosage varies depending on the dosage form employed, condition and sensitivity of the patient, the route of administration, and other factors. The exact dosage will be determined by the practitioner, in light of factors related to the subject that requires treatment. Dosage and administration are adjusted to provide sufficient levels of the active ingredient or to maintain the desired effect. Factors which can be taken into account include the severity of the disease state, general health of the subject, age, weight, and gender of the subject, diet, time and frequency of administration, drug combination(s), reaction sensitivities, and tolerance/response to therapy. During a treatment cycle, the daily dose could be varied. In some embodiments, a starting dosage can be titrated down within a treatment cycle. In some embodiments, a starting dosage can be titrated up within a treatment cycle. The final dosage can depend on the occurrence of dose limiting toxicity and other factors. In some embodiments, the FTI is administered at a starting dose of 300 mg daily and escalated to a maximum dose of 400 mg, 500 mg, 600 mg, 700 mg, 800 mg, 900 mg, 1000 mg, 1100 mg, or 1200 mg daily. In some embodiments, the FTI is administered at a starting dose of 400 mg daily and escalated to a maximum dose of 500 mg, 600 mg, 700 mg, 800 mg, 900 mg, 1000 mg, 1100 mg, or 1200 mg daily. In some embodiments, the FTI is administered at a starting dose of 500 mg daily and escalated to a maximum dose of 600 mg, 700 mg, 800 mg, 900 mg, 1000 mg, 1100 mg, or 1200 mg daily. In some embodiments, the FTI is administered at a starting dose of 600 mg daily and escalated to a maximum dose of 700 mg, 800 mg, 900 mg, 1000 mg, 1100 mg, or 1200 mg daily. In some embodiments, the FTI is administered at a starting dose of 700 mg daily and escalated to a maximum dose of 800 mg, 900 mg, 1000 mg, 1100 mg, or 1200 mg daily. In some embodiments, the FTI is administered at a starting dose of 800 mg daily and escalated to a maximum dose of 900 mg, 1000 mg, 1100 mg, or 1200 mg daily. In some embodiments, the FTI is administered at a starting dose of 900 mg daily and escalated to a maximum dose of 1000 mg, 1100 mg, or 1200 mg daily. The dose escalation can be done at once, or step wise. For example, a starting dose at 600 mg daily can be escalated to a final dose of 1000 mg daily by increasing by 100 mg per day over the course of 4 days, or by increasing by 200 mg per day over the course of 2 days, or by increasing by 400 mg at once. In some embodiments, the FTI is tipifarnib.

In some embodiments, the FTI is administered at a relatively high starting dose and titrated down to a lower dose depending on the patient response and other factors. In some embodiments, the FTI is administered at a starting dose of 1200 mg daily and reduced to a final dose of 1100 mg, 1000 mg, 900 mg, 800 mg, 700 mg, 600 mg, 500 mg, 400 mg, or 300 mg daily. In some embodiments, the FTI is administered at a starting dose of 1100 mg daily and reduced to a final dose of 1000 mg, 900 mg, 800 mg, 700 mg, 600 mg, 500 mg, 400 mg, or 300 mg daily. In some embodiments, the FTI is administered at a starting dose of 1000 mg daily and reduced to a final dose of 900 mg, 800 mg, 700 mg, 600 mg, 500 mg, 400 mg, or 300 mg daily. In some embodiments, the FTI is administered at a starting dose of 900 mg daily and reduced to a final dose of 800 mg, 700 mg, 600 mg, 500 mg, 400 mg, or 300 mg daily. In some embodiments, the FTI is administered at a starting dose of 800 mg daily and reduced to a final dose of 700 mg, 600 mg, 500 mg, 400 mg, or 300 mg daily. In some embodiments, the FTI is administered at a starting dose of 600 mg daily and reduced to a final dose of 500 mg, 400 mg, or 300 mg daily. The dose reduction can be done at once, or step wise. In some embodiments, the FTI is tipifarnib. For example, a starting dose at 900 mg daily can be reduced to a final dose of 600 mg daily by decreasing by 100 mg per day over the course of 3 days, or by decreasing by 300 mg at once.

A treatment cycle can have different length. In some embodiments, a treatment cycle can be one week, 2 weeks, 3 weeks, 4 weeks, 5 weeks, 6 weeks, 7 weeks, 8 weeks, 3 months, 4 months, 5 months, 6 months, 7 months, 8 months, 9 months, 10 months, 11 months, or 12 months. In some embodiments, a treatment cycle is 4 weeks. A treatment cycle can have intermittent schedule. In some embodiments, a 2-week treatment cycle can have 5-day dosing followed by 9-day rest. In some embodiments, a 2-week treatment cycle can have 6-day dosing followed by 8-day rest. In some embodiments, a 2-week treatment cycle can have 7-day dosing followed by 7-day rest. In some embodiments, a 2-week treatment cycle can have 8-day dosing followed by 6-day rest. In some embodiments, a 2-week treatment cycle can have 9-day dosing followed by 5-day rest.

In some embodiments, the FTI is administered daily for 3 of out of 4 weeks in repeated 4 week cycles. In some embodiments, the FTI is administered daily in alternate weeks (one week on, one week off) in repeated 4 week cycles. In some embodiments, the FTI is administered at a dose of 300 mg b.i.d. orally for 3 of out of 4 weeks in repeated 4 week cycles. In some embodiments, the FTI is administered at a dose of 600 mg b.i.d. orally for 3 of out of 4 weeks in repeated 4 week cycles. In some embodiments, the FTI is administered at a dose of 900 mg b.i.d. orally in alternate weeks (one week on, one week off) in repeated 4 week cycles. In some embodiments, the FTI is administered at a dose of 1200 mg b.i.d. orally in alternate weeks (days 1-7 and 15-21 of repeated 28-day cycles). In some embodiments, the FTI is administered at a dose of 1200 mg b.i.d. orally for days 1-5 and 15-19 out of repeated 28-day cycles.

In some embodiments, a 900 mg bid tipifarnib alternate week regimen can be used adopted. Under the regimen, patients receive a starting dose of 900 mg, po, bid on days 1-7 and 15-21 of 28-day treatment cycles. In the absence of unmanageable toxicities, subjects can continue to receive the tipifarnib treatment for up to 12 months. The dose can also be increased to 1200 mg bid if the subject is tolerating the treatment well. Stepwise 300 mg dose reductions to control treatment-related, treatment-emergent toxicities can also be included.

In some other embodiments, tipifarnib is given orally at a dose of 300 mg bid daily for 21 days, followed by 1 week of rest, in 28-day treatment cycles (21-day schedule; Cheng D T, et al., J Mol Diagn. (2015) 17(3):251-64). In some embodiments, a 5-day dosing ranging from 25 to 1300 mg bid followed by 9-day rest is adopted (5-day schedule; Zujewski J., J Clin Oncol., (2000) February; 18(4):927-41). In some embodiments, a 7-day bid dosing followed by 7-day rest is adopted (7-day schedule; Lara P N Jr., Anticancer Drugs., (2005) 16(3):317-21; Kirschbaum M H, Leukemia., (2011) October; 25(10):1543-7). In the 7-day schedule, the patients can receive a starting dose of 300 mg bid with 300 mg dose escalations to a maximum planned dose of 1800 mg bid. In the 7-day schedule study, patients can also receive tipifarnib bid on days 1-7 and days 15-21 of 28-day cycles at doses up to 1600 mg bid.

In previous studies FTI were shown to inhibit the growth of mammalian tumors when administered as a twice daily dosing schedule. It was found that administration of an FTI in a single dose daily for one to five days produced a marked suppression of tumor growth lasting out to at least 21 days. In some embodiments, FTI is administered at a dosage range of 50-400 mg/kg. In some embodiments, FTI is administered at 200 mg/kg. Dosing regimen for specific FTIs are also well known in the art (e.g., U.S. Pat. No. 6,838,467, which is incorporated herein by reference in its entirety). For example, suitable dosages for the compounds Arglabin (WO98/28303), perrilyl alcohol (WO 99/45712), SCH-66336 (U.S. Pat. No. 5,874,442), L778123 (WO 00/01691), 2(S)-[2(S)-[2(R)-amino-3-mercapto]propylamino-3(S)-methyl]-pentyloxy-3-phenylpropionyl-methionine sulfone (WO94/10138), BMS 214662 (WO 97/30992), AZD3409; Pfizer compounds A and B (WO 00/12499 and WO 00/12498) are given in the aforementioned patent specifications which are incorporated herein by reference or are known to or can be readily determined by a person skilled in the art.

In relation to perrilyl alcohol, the medicament may be administered 1-4 g per day per 150 lb human patient. Preferably, 1-2 g per day per 150 lb human patient. SCH-66336 typically may be administered in a unit dose of about 0.1 mg to 100 mg, more preferably from about 1 mg to 300 mg according to the particular application. Compounds L778123 and 1-(3-chlorophenyl)-4-[1-(4-cyanobenzyl)-5-imidazolylmethyl]-2-piperazinone may be administered to a human patient in an amount between about 0.1 mg/kg of body weight to about 20 mg/kg of body weight per day, preferably between 0.5 mg/kg of bodyweight to about 10 mg/kg of body weight per day.

Pfizer compounds A and B may be administered in dosages ranging from about 1.0 mg up to about 500 mg per day, preferably from about 1 to about 100 mg per day in single or divided (i.e. multiple) doses. Therapeutic compounds will ordinarily be administered in daily dosages ranging from about 0.01 to about 10 mg per kg body weight per day, in single or divided doses. BMS 214662 may be administered in a dosage range of about 0.05 to 200 mg/kg/day, preferably less than 100 mg/kg/day in a single dose or in 2 to 4 divided doses.

In some embodiments, the FTI treatment is administered in combination with radiotherapy, or radiation therapy. Radiotherapy includes using γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287; all of which are hereby incorporated by references in their entireties), and UV-irradiation. It is most likely that all of these factors affect a broad range of damage on DNA, on the precursors of DNA, on the replication and repair of DNA, and on the assembly and maintenance of chromosomes.

In some embodiments, a therapeutically effective amount of the pharmaceutical composition having an FTI is administered that effectively sensitizes a tumor in a host to irradiation. (U.S. Pat. No. 6,545,020, which is hereby incorporated by reference in its entirety). Irradiation can be ionizing radiation and in particular gamma radiation. In some embodiments, the gamma radiation is emitted by linear accelerators or by radionuclides. The irradiation of the tumor by radionuclides can be external or internal.

Irradiation can also be X-ray radiation. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

In some embodiments, the administration of the pharmaceutical composition commences up to one month, in particular up to 10 days or a week, before the irradiation of the tumor. Additionally, irradiation of the tumor is fractionated the administration of the pharmaceutical composition is maintained in the interval between the first and the last irradiation session.

The amount of FTI, the dose of irradiation and the intermittence of the irradiation doses will depend on a series of parameters such as the type of tumor, its location, the patients' reaction to chemo- or radiotherapy and ultimately is for the physician and radiologists to determine in each individual case.

In some embodiments, the methods provided herein further include administering a therapeutically effective amount of a second active agent or a support care therapy. The second active agent can be a chemotherapeutic agent. A chemotherapeutic agent or drug can be categorized by its mode of activity within a cell, for example, whether and at what stage they affect the cell cycle. Alternatively, an agent can be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gamma1I and calicheamicin omegaI1); dynemicin, including dynemicin A; bisphosphonates, such as clodronate; an esperamicin; as well as neocarzinostatin chromophore and related chromoprotein enediyne antibiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabine, navelbine, transplatinum, and pharmaceutically acceptable salts, acids, or derivatives of any of the above.

The second active agents can be large molecules (e.g., proteins) or small molecules (e.g., synthetic inorganic, organometallic, or organic molecules). In some embodiments, the second active agent is a DNA-hypomethylating agent, a therapeutic antibody that specifically binds to a cancer antigen, a hematopoietic growth factor, cytokine, anti-cancer agent, antibiotic, cox-2 inhibitor, immunomodulatory agent, anti-thymocyte globulin, immunosuppressive agent, corticosteroid or a pharmacologically active mutant or derivative thereof.

In some embodiments, the second active agent is a DNA hypomethylating agent, such as a cytidine analog (e.g., azacitidine) or a 5-azadeoxycytidine (e.g. decitabine). In some embodiments, the second active agent is a cytoreductive agent, including but not limited to Induction, Topotecan, Hydrea, PO Etoposide, Lenalidomide, LDAC, and Thioguanine. In some embodiments, the second active agent is Mitoxantrone, Etoposide, Cytarabine, or Valspodar. In some embodiment, the second active agent is Mitoxantrone plus Valspodar, Etoposide plus Valspodar, or Cytarabine plus Valspodar. In some embodiment, the second active agent is idarubicin, fludarabine, topotecan, or ara-C. In some other embodiments, the second active agent is idarubicin plus ara-C, fludarabine plus ara-C, mitoxantrone plus ara-C, or topotecan plus ara-C. In some embodiments, the second active agent is a quinine. Other combinations of the agents specified above can be used, and the dosages can be determined by the physician.

For any specific cancer type described herein, treatments as described herein or otherwise available in the art can be used in combination with the FTI treatment. For example, drugs that can be used in combination with the FTI for MDS include belinostat (Beleodaq®) and pralatrexate (Folotyn®), marketed by Spectrum Pharmaceuticals, romidepsin)(Istodax®), marketed by Celgene, and brentuximab vedotin (Adcetris®) (for ALCL), marketed by Seattle Genetics; drugs that can be used in combination with the FTI for MDS include azacytidine (Vidaza®) and lenalidomide (Revlimid®), marketed by Celgene, and decitabine (Dacogen®) marketed by Otsuka and Johnson & Johnson; drugs that can be used in combination with the FTI for thyroid cancer include AstraZeneca's vandetanib (Caprelsa®), Bayer's sorafenib (Nexavar®), Exelixis' cabozantinib (Cometriq®) and Eisai's lenvatinib (Lenvima®).

Non-cytotoxic therapies such as tpralatrexate (Folotyn®), romidepsin (Istodax®) and belinostat (Beleodaq®) can also be used in combination with the FTI treatment.

In some embodiments, it is contemplated that the second active agent or second therapy used in combination with a FTI can be administered before, at the same time, or after the FTI treatment. In some embodiments, the second active agent or second therapy used in combination with a FTI can be administered before the FTI treatment. In some embodiments, the second active agent or second therapy used in combination with a FTI can be administered at the same time as FTI treatment. In some embodiments, the second active agent or second therapy used in combination with a FTI can be administered after the FTI treatment.

The FTI treatment can also be administered in combination with a bone marrow transplant. In some embodiments, the FTI is administered before the bone marrow transplant. In other embodiments, the FTI is administered after the bone marrow transplant.

A person of ordinary skill in the art would understand that the methods described herein include using any permutation or combination of the specific FTI, formulation, dosing regimen, additional therapy to treat MDS in a subject characterized by Th1 dominance. In some embodiments, the MDS can be lower risk MDS.

In some embodiments, provided herein are methods for predicting responsiveness of a subject having MDS to a tipifarnib treatment, methods for MDS patient population selection for a tipifarnib treatment, and methods for treating MDS in a subject with a therapeutically effective amount of tipifarnib, by determining that a MDS patient has Th1 dominance, or selecting MDS patients characterized by Th1 dominance. In some embodiments, the method includes determining by qRT-PCR that the TBX21 expression level in a tumor sample from the subject having MDS is higher than a reference level, and subsequently administering a therapeutically effective amount of tipifarnib to the subject. In some embodiments, the method includes determining by qRT-PCR that the ratio of TBX21 expression level to the GATA3 expression level in a tumor sample from the subject having MDS is higher than a reference ratio, and subsequently administering a therapeutically effective amount of tipifarnib to the subject. In some embodiments, the method includes determining by FACS the ratio of Th1 cells to Th2 cells in the subject having MDS is higher than a reference ratio, and subsequently administering a therapeutically effective amount of tipifarnib to the subject. In some embodiments, the method includes determining by FACS that the percentage of Th1 cells in a tumor sample from the subject having MDS is higher than a reference percentage, and subsequently administering a therapeutically effective amount of tipifarnib to the subject. In some embodiments, the method includes determining by ELISA that the level of IFN-γ in a serum sample from the subject having MDS is higher than a reference ratio, and subsequently administering a therapeutically effective amount of tipifarnib to the subject. In some embodiments, the method includes determining by ELISA that the ratio of IFN-γ level to IL-4 level in the subject having MDS is higher than a reference ratio, and subsequently administering a therapeutically effective amount of tipifarnib to the subject. In some embodiments, the MDS can be lower risk MDS.

In some embodiments, the subject having MDS who is selected from tipifarnib treatment receives a dose of 900 mg b.i.d. orally in alternate weeks (one week on, one week off) in repeated 4 week cycles.

Provided herein are also kits for predicting the responsiveness of a subject having MDS to an FTI treatment. The kits provided herein can include an ancillary agent. In some embodiments, the kits include an agent for determining the expression level of a Th1 gene signature in a sample from a subject having MDS, wherein the subject having MDS is predicted to be responsive to the FTI treatment if the expression level of the Th1 gene signature is higher than a reference expression level of said Th1 gene signature. In some embodiments, the kits include an agent for determining the ratio of Th1 cells to Th2 cells in a sample from a subject having MDS, wherein the subject is predicted to be responsive to the FTI treatment if the ratio of Th1 cells to Th2 cells in the sample is higher than a reference ratio. In some embodiments, the kit include an agent for detecting a Th1 cytokine in a sample from a subject having MDS, wherein the subject is predicted to be responsive to the FTI treatment if the Th1 cytokine is present in the sample.

In some embodiments, the kits provided herein can also include an ancillary agent. In some embodiments, the kits further include reagents for genomic DNA isolation or purification means, detection means, as well as positive and negative controls. In certain embodiments, the kits further include instructions for users. In some embodiments, the kits further includes an FTI or a pharmaceutical composition having an FTI. The kits can be tailored for in-home use, clinical use, or research use.

In some embodiments, the kits provided herein include an agent for determining the expression level of a Th1 gene signature. The Th1 gene signature can be TBX21, STAT1, STAT6, CXCR3, CCR5, IFN-γ, TNF-α, IL-2, IL-12, or any combination thereof. In some embodiments, kits provided herein include agents for determining the expression levels of at least two, three, four, five, six, seven, eight, or nine Th1 gene signatures. In some embodiments, the kits include an agent for determining the expression level of TBX21. In some embodiments, the kits further include an agent for determining the expression level of a Th2 gene signature in a sample from a subject having MDS, wherein the subject is predicted to be responsive to the FTI treatment if the ratio is higher than a reference ratio. The Th2 gene signature can be GATA3, CCR4, IL-4, IL-5, IL-6, IL-10, IL-13, or any combination thereof. In some embodiments, the Th2 gene signature is GATA3. In some embodiments, the kits include agents for determining the expression level of TBX21 and GATA3 in a sample from a subject having MDS, wherein the subject is predicted to be responsive to the FTI treatment if the ratio is higher than a reference ratio.

In certain embodiments, provided herein is a kit including an agent for detecting the mRNA level of one or more Th1 gene signature. In certain embodiments, the agent can be one or more probes that bind specifically to the mRNAs of the one or more Th1 gene signatures. In certain embodiments, the kit further includes a washing solution. In certain embodiments, the kit further includes reagents for performing a hybridization assay, mRNA isolation or purification means, detection means, as well as positive and negative controls. In certain embodiments, the kit further includes an instruction for using the kit. In some embodiments, the kit further includes an FTI or a pharmaceutical composition having an FTI. The kit can be tailored for in-home use, clinical use, or research use.

In certain embodiments, provided herein is a kit including an agent for detecting the protein level of one or more Th1 gene signatures. In certain embodiments, the kits includes a dipstick coated with an antibody that recognizes the Th1 gene signature, washing solutions, reagents for performing the assay, protein isolation or purification means, detection means, as well as positive and negative controls. In certain embodiments, the kit further includes an instruction for using the kit. In some embodiments, the kit further includes an FTI or a pharmaceutical composition having an FTI. The kit can be tailored for in-home use, clinical use, or research use.

In some embodiments, the kits include an agent for determining the ratio of Th1 cells to Th2 cells in a sample from a subject having MDS, wherein the subject is predicted to be responsive to the FTI treatment if the ratio of Th1 cells to Th2 cells in the sample is higher than a reference ratio. The agent included in the kit can be a reagent required to conduct an immunohistochemistry (IHC) assay, an immunofluorescence (IF) assay, or flow cytometry (FACS) to measure the numbers of Th1 cells and Th2 cells in a sample. The agent can be an agent to detect or determine the expression level of one or more Th1 gene signatures or Th2 gene signatures as described above. In some embodiments, the kits described herein include an agent to perform an IHC assay to determine the ratio of Th1 cells to Th2 cells in a sample. The agent can include an antibody recognizing the product of a Th1 gene signature and an antibody recognizing the product of a Th2 gene signature. In some embodiments, the kits provided herein include an antibody recognizing TBX21 and an antibody recognizing GATA3. In some embodiments, the kits described herein include an agent to perform an FACS analysis to determine the ratio of Th1 cells to Th2 cells in a sample. The agent can include an antibody recognizing the product of a Th1 gene signature and an antibody recognizing the product of a Th2 gene signature. In some embodiments, the kits provided herein include an antibody recognizing TBX21 and an antibody recognizing GATA3. The agent can also include an antibody recognizing the product of a Th1 cell surface marker and an antibody recognizing the product of a Th2 cell surface marker. The Th1 cell surface marker can be, for example, CD4 and CXCR3. The Th2 cell surface marker can be, for example, CD4 and CCR4.

In some embodiments, the kits provided herein include an agent for detecting a Th1 cytokine in a sample from a subject having MDS, wherein the subject is predicted to be responsive to the FTI treatment if the Th1 cytokine is present in the sample. In some embodiments, the kits provided herein further include an agent for detecting a Th2 cytokine in a sample from a subject having MDS, wherein the subject is predicted to be responsive to the FTI treatment if the Th2 cytokine is absent in the sample, or if the ratio of the level of a Th1 cytokine to that of a Th2 cytokine is higher than a reference ratio. The agent included in the kit can be a reagent required to conduct an IHC assay, an IB assay, an IF assay, FACS, ELISA, protein microarray analysis, qPCR, qRT-PCR, RNA-seq, RNA microarray analysis, SAGE, MassARRAY technique, next-generation sequencing, or FISH to detect one or more Th1 cytokines and/or one or more Th2 cytokines. In some embodiments, the agent included in the kit can also be a reagent required to conduct RT-PCR, microarray, FACS, ELISA, Cytometric Bead Array (“CBA”), or Intracellular cytokine staining (ICS) to detect one or more Th1 cytokines and/or one or more Th2 cytokines.

The kits provided herein can employ, for example, a dipstick, a membrane, a chip, a disk, a test strip, a filter, a microsphere, a slide, a multiwell plate, or an optical fiber. The solid support of the kit can be, for example, a plastic, silicon, a metal, a resin, glass, a membrane, a particle, a precipitate, a gel, a polymer, a sheet, a sphere, a polysaccharide, a capillary, a film, a plate, or a slide. The sample can be, for example, a blood sample, a bone marrow sample, a cell culture, a cell line, a tissue, an oral issue, gastrointestinal tissue, an organ, an organelle, a biological fluid, a urine sample, or a skin sample. The biological sample can be, for example, a lymph node biopsy, a bone marrow biopsy, or a sample of peripheral blood tumor cells.

In some embodiments, the kits provided herein include one or more containers and components for conducting RT-PCR, qPCR, deep sequencing, NGS, or a microarray. In certain embodiments, the kits provided herein employ means for detecting the expression of a gene signature by flow cytometry or immunofluorescence. In other embodiments, the expression of the gene signature is measured by ELISA-based methodologies or other similar methods known in the art.

In certain embodiments, the kits provided herein include components for isolating protein. In another specific embodiment, the pharmaceutical or assay kit includes, in a container, an FTI or a pharmaceutical composition having an FTI, and further includes, in one or more containers, components for conducting flow cytometry or an ELISA.

In some embodiments, provided herein are kits for measuring gene signatures providing the materials necessary to measure the presence of certain genes, or abundance of one or more of the gene products of the genes or a subset of genes (e.g., one, two, three, four, five or more genes) of the gene signatures provided herein. Such kits can include materials and reagents required for measuring DNA, RNA or protein. In some embodiments, such kits include microarrays, wherein the microarray is comprised of oligonucleotides and/or DNA and/or RNA fragments which hybridize to one or more of the DNA or mRNA transcripts of one or more of the gene signatures or a subset of gene signatures provided herein, or any combination thereof. In some embodiments, such kits can include primers for PCR of either the DNA, RNA product or the cDNA copy of the RNA product of the genes or subset of genes. In some embodiments, such kits can include primers for PCR as well as probes for Quantitative PCR. In some embodiments, such kits can include multiple primers and multiple probes wherein some of the probes have different fluorophores so as to permit multiplexing of multiple products of a gene product or multiple gene products. In some embodiments, such kits can further include materials and reagents for synthesizing cDNA from RNA isolated from a sample. In some embodiments, such kits can include antibodies specific for the protein products of a gene signature or subset of gene signatures provided herein. Such kits can additionally include materials and reagents for isolating RNA and/or proteins from a biological sample. In some embodiments, such kits can include, a computer program product embedded on computer readable media for predicting whether a patient is clinically sensitive to an FTI. In some embodiments, the kits can include a computer program product embedded on a computer readable media along with instructions.

In some embodiments, kits for measuring the expression of one or more nucleic acid sequences of a gene signature or a subset of gene signatures provided herein. In a specific embodiment, such kits measure the expression of one or more nucleic acid sequences associated with a gene signature or a subset of gene signatures provided herein. In accordance with this embodiment, the kits can include materials and reagents that are necessary for measuring the expression of particular nucleic acid sequence products of gene signatures or a subset of gene signatures provided herein. For example, a microarray or RT-PCR kit can be produced for a specific condition and contain only those reagents and materials necessary for measuring the levels of specific RNA transcript products of the gene signatures or a subset of gene signatures provided herein to predict whether a subject having MDS would be clinically sensitive to an FTI. Alternatively, in some embodiments, the kits can include materials and reagents that are not limited to those required to measure the expression of particular nucleic acid sequences of any particular gene signatures provided herein. For example, in certain embodiments, the kits comprise materials and reagents necessary for measuring the levels of expression of 1, 2, 3, 4, or 5 of the gene signatures provided herein, in addition to reagents and materials necessary for measuring the levels of the expression of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50 or more genes other than those of the gene signatures provided herein. In other embodiments, the kits contain reagents and materials necessary for measuring the levels of expression of at least 1, at least 2, at least 3, at least 4, at least 5, or more of the gene signatures provided herein, and 1, 2, 3, 4, 5, 10, 15, 20, 25, 30, 35, 40, 45, 50, 55, 60, 65, 70, 75, 80, 85, 90, 95, 100, 125, 150, 175, 200, 225, 250, 300, 350, 400, 450, or more genes that are genes not of the gene signatures provided herein, or 1-10, 1-100, 1-150, 1-200, 1-300, 1-400, 1-500, 1-1000, 25-100, 25-200, 25-300, 25-400, 25-500, 25-1000, 100-150, 100-200, 100-300, 100-400, 100-500, 100-1000 or 500-1000 genes that are genes not of the gene signatures provided herein.

For nucleic acid microarray kits, the kits generally include probes attached to a solid support surface. In one such embodiment, probes can be either be oligonucleotides or longer length probes including probes ranging from 150 nucleotides in length to 800 nucleotides in length. The probes can be attached to a detectable label. In a specific embodiment, the probes are specific for one or more of the products of the gene signatures provided herein. The microarray kits can include instructions for performing the assay and methods for interpreting and analyzing the data resulting from the performance of the assay. In a specific embodiment, the kits include instructions for predicting whether a subject having MDS would be clinically sensitive to an FTI. The kits can also include hybridization reagents and/or reagents necessary for detecting a signal produced when a probe hybridizes to a target nucleic acid sequence. Generally, the materials and reagents for the microarray kits are in one or more containers. Each component of the kit is generally in its own a suitable container.

In certain embodiments, a nucleic acid microarray kit includes materials and reagents necessary for measuring the levels of expression of 1, 2, 3, 4, 5, 6, 7, 8, 9, 10, 15, 20, 25, 30, 35, 40, 45, 50 or more of the gene signatures provided herein, or a combination thereof, in addition to reagents and materials necessary for measuring the levels of the expression of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50 or more genes other than those of the gene signatures provided herein. In other embodiments, a nucleic acid microarray kit contains reagents and materials necessary for measuring the levels of expression of at least 1, at least 2, at least 3, at least 4, at least 5, at least 6, at least 7, at least 8, at least 9, at least 10, at least 15, at least 20, at least 25, at least 30, at least 35, at least 40, at least 45, at least 50 or more of the gene signatures provided herein, or any combination thereof, and 1, 2, 3, 4, 5, 10, 15, 20, 25, 30, 35, 40, 45, 50, 55, 60, 65, 70, 75, 80, 85, 90, 95, 100, 125, 150, 175, 200, 225, 250, 300, 350, 400, 450, or more genes that are not of the gene signatures provided herein, or 1-10, 1-100, 1-150, 1-200, 1-300, 1-400, 1-500, 1-1000, 25-100, 25-200, 25-300, 25-400, 25-500, 25-1000, 100-150, 100-200, 100-300, 100-400, 100-500, 100-1000 or 500-1000 genes that are not of the gene signatures provided herein.

For Quantitative PCR, the kits can include pre-selected primers specific for particular nucleic acid sequences. The Quantitative PCR kits can also include enzymes suitable for amplifying nucleic acids (e.g., polymerases such as Taq), and deoxynucleotides and buffers needed for the reaction mixture for amplification. The Quantitative PCR kits can also include probes specific for the nucleic acid sequences associated with or indicative of a condition. The probes can be labeled with a fluorophore. The probes can also be labeled with a quencher molecule. In some embodiments the Quantitative PCR kits can also include components suitable for reverse-transcribing RNA including enzymes (e.g., reverse transcriptases such as AMV, MMLV and the like) and primers for reverse transcription along with deoxynucleotides and buffers needed for the reverse transcription reaction. Each component of the quantitative PCR kit is generally in its own suitable container. Thus, these kits generally include distinct containers suitable for each individual reagent, enzyme, primer and probe. Further, the quantitative PCR kits can include instructions for performing the assay and methods for interpreting and analyzing the data resulting from the performance of the assay. In a specific embodiment, the kits contain instructions for predicting whether a subject having MDS would be clinically sensitive to an FTI.

For antibody based kits, the kit can include, for example: (1) a first antibody which binds to a polypeptide or protein of interest; and, optionally, (2) a second, different antibody which binds to either the polypeptide or protein, or the first antibody and is conjugated to a detectable label (e.g., a fluorescent label, radioactive isotope or enzyme). The first antibody can be attached to a solid support. In a specific embodiment, the polypeptide or protein of interest is a gene signature provided herein. The antibody-based kits can also include beads for conducting an immunoprecipitation. Each component of the antibody-based kits is generally in its own suitable container. Thus, these kits generally include distinct containers suitable for each antibody. Further, the antibody-based kits can include instructions for performing the assay and methods for interpreting and analyzing the data resulting from the performance of the assay. In a specific embodiment, the kits contain instructions for predicting whether a subject having MDS is clinically sensitive to an FTI.

In some embodiments a kit provided herein includes an FTI provided herein, or a pharmaceutically composition having an FTI. Kits can further include additional active agents, including but not limited to those disclosed herein, such as a DNA-hypomethylating agent, a therapeutic antibody that specifically binds to a cancer antigen, a hematopoietic growth factor, a cytokine, an anti-cancer agent, an antibiotic, a cox-2 inhibitor, an immunomodulatory agent, an anti-thymocyte globulin, an immunosuppressive agent, or a corticosteroid.

Kits provided herein can further include devices that are used to administer the FTI or other active ingredients. Examples of such devices include, but are not limited to, syringes, drip bags, patches, and inhalers.

Kits can further include cells or blood for transplantation as well as pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

In certain embodiments of the methods and kits provided herein, solid phase supports are used for purifying proteins, labeling samples or carrying out the solid phase assays. Examples of solid phases suitable for carrying out the methods disclosed herein include beads, particles, colloids, single surfaces, tubes, multiwell plates, microtiter plates, slides, membranes, gels and electrodes. When the solid phase is a particulate material (e.g., beads), it is, in one embodiment, distributed in the wells of multi-well plates to allow for parallel processing of the solid phase supports.

The kit of this disclosure can include an ancillary reagent. In some embodiments, the ancillary reagent can be a secondary antibody, a detection reagent, a detection buffer, an immobilization buffer, a dilution buffer, a washing buffer, or any combination thereof.

Secondary antibodies can be monoclonal or polyclonal antibodies. Secondary antibodies can be derived from any mammalian organism, including bovine, mice, rats, hamsters, goats, camels, chicken, rabbit, and others. Secondary antibodies can include, for example, an anti-human IgA antibody, an anti-human IgD antibody, an anti-human IgE antibody, an anti-human IgG antibody, or an anti-human IgM antibody. Secondary antibodies can be conjugated to enzymes (e.g., horseradish peroxidase (HRP), alkaline phosphatase (AP), luciferase, and the like) or dyes (e.g., colorimetric dyes, fluorescent dyes, fluorescence resonance energy transfer (FRET)-dyes, time-resolved (TR)-FRET dyes, and the like). In some embodiments, the secondary antibody is a polyclonal rabbit-anti-human IgG antibody, which is HRP-conjugated.

Any detection reagent known in the art can be included in a kit of this disclosure. In some embodiments, the detection reagent is a colorimetric detection reagent, a fluorescent detection reagent, or a chemiluminescent detection reagent. In some embodiments, the colorimetric detection reagent includes PNPP (p-nitrophenyl phosphate), ABTS (2,2′-azino-bis(3-ethylbenzothiazoline-6-sulphonic acid)) or OPD (o-phenylenediamine). In some embodiments, the fluorescent detection reagent includes QuantaBlu™ or QuantaRed™ (Thermo Scientific, Waltham, Mass.). In some embodiments, the luminescent detection reagent includes luminol or luciferin. In some embodiments, the detection reagent includes a trigger (e.g., H2O2) and a tracer (e.g., isoluminol-conjugate).

Any detection buffer known in the art can be included in a kit of this disclosure. In some embodiments the detection buffer is a citrate-phosphate buffer (e.g., about pH 4.2).

Any stop solution known in the art can be included in a kit of this disclosure. The stop solutions of this disclosure terminate or delay the further development of the detection reagent and corresponding assay signals. Stop solutions can include, for example, low-pH buffers (e.g., glycine-buffer, pH 2.0), chaotrophic agents (e.g., guanidinium chloride, sodium-dodecylsulfate (SDS)) or reducing agents (e.g., dithiothreitol, mecaptoethanol), or the like.

In some embodiments, the ancillary reagent is an immobilization reagent, which can be any immobilization reagent known in the art, including covalent and non-covalent immobilization reagents. Covalent immobilization reagents can include any chemical or biological reagent that can be used to covalently immobilize a peptide or a nucleic acid on a surface. Covalent immobilization reagents can include, for example, a carboxyl-to-amine reactive group (e.g., carbodiimides such as EDC or DCC), an amine reactive group (e.g., N-hydroxysuccinimide (NHS) esters, imidoesters), a sulfhydryl-reactive crosslinker (e.g., maleimides, haloacetyls, pyridyl disulfides), a carbonyl-reactive crosslinker groups (e.g., hydrazides, alkoxyamines), a photoreactive crosslinker (e.g., aryl azides, dizirines), or a chemoselective ligation group (e.g., a Staudinger reaction pair). Non-covalent immobilization reagents include any chemical or biological reagent that can be used to immobilize a peptide or a nucleic acid non-covalently on a surface, such as affinity tags (e.g., biotin) or capture reagents (e.g., streptavidin or anti-tag antibodies, such as anti-His6 or anti-Myc antibodies).

The kits of this disclosure can include combinations of immobilization reagents. Such combinations include, for example, EDC and NHS, which can be used, for example, to immobilize a protein of this disclosure on a surface, such as a carboxylated dextrane matrix (e.g., on a BIAcore™ CM5 chip or a dextrane-based bead). Combinations of immobilization reagents can be stored as premixed reagent combinations or with one or more immobilization reagents of the combination being stored separately from other immobilization reagents.

A large selection of washing buffers are known in the art, such as tris(hydroxymethyl)aminomethane (Tris)-based buffers (e.g., Tris-buffered saline, TBS) or phosphate buffers (e.g., phosphate-buffered saline, PBS). Washing buffers can include detergents, such as ionic or non-ionic detergents. In some embodiments, the washing buffer is a PBS buffer (e.g., about pH 7.4) including Tween®20 (e.g., about 0.05% Tween®20).

Any dilution buffer known in the art can be included in a kit of this disclosure. Dilution buffers can include a carrier protein (e.g., bovine serum albumin, BSA) and a detergent (e.g., Tween®20). In some embodiments, the dilution buffer is PBS (e.g., about pH 7.4) including BSA (e.g., about 1% BSA) and Tween®20 (e.g., about 0.05% Tween®20).

In some embodiments, the kit of this disclosure includes a cleaning reagent for an automated assay system. An automated assay system can include systems by any manufacturer. In some embodiments, the automated assay systems include, for example, the BIO-FLASH™, the BEST 2000™, the DS2™, the ELx50 WASHER, the ELx800 WASHER, and the ELx800 READER. A cleaning reagent can include any cleaning reagent known in the art.

It is noted that any combination of the above-listed embodiments, for example, with respect to one or more reagents, such as, without limitation, nucleic acid primers, solid support and the like, are also contemplated in relation to any of the various methods and/or kits provided herein.

It is understood that modifications which do not substantially affect the activity of the various embodiments of this invention are also provided within the definition of the invention provided herein. Accordingly, the following examples are intended to illustrate but not limit the present invention. All of the references cited to herein are incorporated by reference in their entireties.

### Example I

### Tipifarnib Clinical Study in MDS Patients Based on Serum Th1/Th2 Cytokine Levels

A clinical study of tipifarnib can be conducted with the primary objective being to assess the antitumor activity of tipifarnib, in terms of Objective Response Rate (ORR) in subjects with lower risks MDS who are characterized by high levels of Th1 cytokines and low levels of Th2 cytokines in serum. Determination of objective tumor response can be performed by the International Workshop Criteria (IWC) and/or measurable cutaneous disease according to the modified Severity Weighted Assessment Tool (mSWAT). Secondary objectives can include accessing the effect of tipifarnib on rate of progression-free survival (PFS) at 1 year, duration of response (DOR), overall survival (OS); and safety and tolerability of tipifarnib.

This clinical study investigates the antitumor activity in terms of ORR of tipifarnib in subjects with lower risk MDS. Up to 18 eligible subjects with advanced MDS are enrolled. The total number of patients can be extended to 30.

Subjects receive tipifarnib administered at a starting dose of 900 mg, orally with food, twice a day (bid) for 7 days in alternating weeks (Days 1-7 and 15-21) in 28 day cycles. At the discretion of the investigator, the dose of tipifarnib can be increased to 1200 mg bid if the subject has not experienced dose limiting toxicities at the 900 mg dose level. Subjects who develop serious adverse events (SAE) or ≧grade 2 treatment-emergent adverse events (TEAE) that are deemed related to tipifarnib and lasting ≧14 days will not undergo dose escalation. Stepwise 300 mg dose reductions to control treatment-related, treatment-emergent toxicities are also allowed.

In the absence of unmanageable toxicities, subjects can continue to receive tipifarnib treatment until disease progression. If a complete response is observed, therapy with tipifarnib can be maintained for at least 6 months beyond the start of response.

Tumor assessments are performed at screening and at least once every approximately 8 weeks for 6 months (cycles 2, 4, 6) and once every approximately 12 weeks (cycles 9, 12, 15, etc.) thereafter, until disease progression, starting at the end of Cycle 2. Additional tumor assessments can be conducted if deemed necessary by the Investigator. Subjects who discontinue tipifarnib treatment for reasons other than disease progression must continue tumor assessments until disease progression, withdrawal of subject's consent to study

### Example II

### Individualized Treatment Decisions for MDS Patients

The following procedures can be taken to determine whether a MDS patient is suitable for an FTI treatment, such as a tipifarnib treatment.

Immunostaining for TBX21 can be performed on formalin-fixed, paraffin-embedded tissue sections from patients following microwave antigen retrieval in a 1-mmol/L concentration of EDTa, pH 8.0, with a previously described human TBX21 monoclonal antibody (e.g. Finotto et al., Science, 2002; 295:3386-338), using a standard indirect avidin-biotin horseradish peroxidise method and diaminobenzidine color development as is well-known in the art. Cases are regarded as immunoreactive for TBX21 if at least 25% of neoplastic cells exhibited positive staining. TBX21 staining can be compared with that of mouse IgG isotype control anti-body diluted to identical protein concentration for all cases studied, to confirm staining specificity.

T-cells can be isolated from the Peripheral blood mononuclear cells (PBMCs) obtained from patient serum. Total RNA can be extracted from cell samples using the Trizol Kit (Qiagen, Santa Clarita, Calif.). RNA quality can be determined by assessing the presence of ribosomal bands on an Agilent Bioanalyzer (Agilent, Palo Alto, Calif.). Good-quality samples can be used for reverse transcription (RT) reactions using the High Capacity cDNA Reverse Transcription Kit (Applied Biosystems, Foster City, Calif.) according to the manufacturer's instructions. Quantitative RT-PCR (qRT-PCR) can be performed for T-bet (TBX21) and EEF1A1 using the ABI Prism 7900HT Sequence Detection System (Applied Biosystems) with all samples run in triplicate. A negative control without cDNA template can be run with every assay. Transcript copy number per individual can be calculated by normalization to EEF1A1 expression.

Alternatively, immune cytokine profiling can be performed to determine the levels of IFN-γ and IL-4 using techniques well known in the art (e.g. Raziuddin et al., Cancer, 1994: 2426-2431).

If the MDS patient is determined to have TBX21 overexpression, and/or if the MDS patient is determined to have high levels of Th1 cytokine (e.g. IFN-γ) and low levels of Th2 cytokine (e.g. IL-4), and if the patient is not otherwise prevented from receiving a tipifarnib treatment, a tipifarnib treatment is prescribed. On the other hand, if the MDS patient is determined to not have either TBX21 overexpression, or if the MDS patient is determined to have low levels of Th1 cytokine (IFN-γ) or high levels of Th2 cytokine (IL-4), a tipifarnib treatment is not recommended.

If a tipifarnib treatment is prescribed to the MDS patient, the MDS patient can simultaneously receive another treatment, such as ionizing radiation, or a second active agent or a support care therapy, as deemed fit by the oncologist. The second active agent can be a DNA-hypomethylating agent, such as azacitidine or decitabine.

### Example III

### Clinical Study of Tipifarnib in Subjects with Transfusion-Dependent, Very Low, Low, or Intermediate Risk Myelodysplastic Syndromes

A two-stage study was designed and is currently ongoing to investigate the antitumor activity of tipifarnib in approximately 58 eligible subjects with very low, low or Intermediate risk MDS who have no known curative treatment. Eligible subjects may have received no more than two prior systemic therapies. In the first stage, 44 eligible subjects will be enrolled and stratified in to one of four biomarker-defined strata (11 subjects per stratum) based on subject KIR2DS2 and KIR2DL2 positively. Subjects receive tipifarnib administered at a starting dose of 900 mg, orally with food, twice a day (bid) for 7 days in alternating weeks (Days 1-7 and 15-21) in 28 day cycles. At the discretion of the investigator, the dose of tipifarnib may be increased to 1200 mg bid if the subject has not experienced dose limiting toxicities at the 900 mg dose level.

Determination of RBC tranfusion independence and disease response are performed by the Investigator according to the MDS/MPN International Working Group (IWG) criteria. Similarly, disease progression are also determined based on the MDS/MPN IWG criteria.

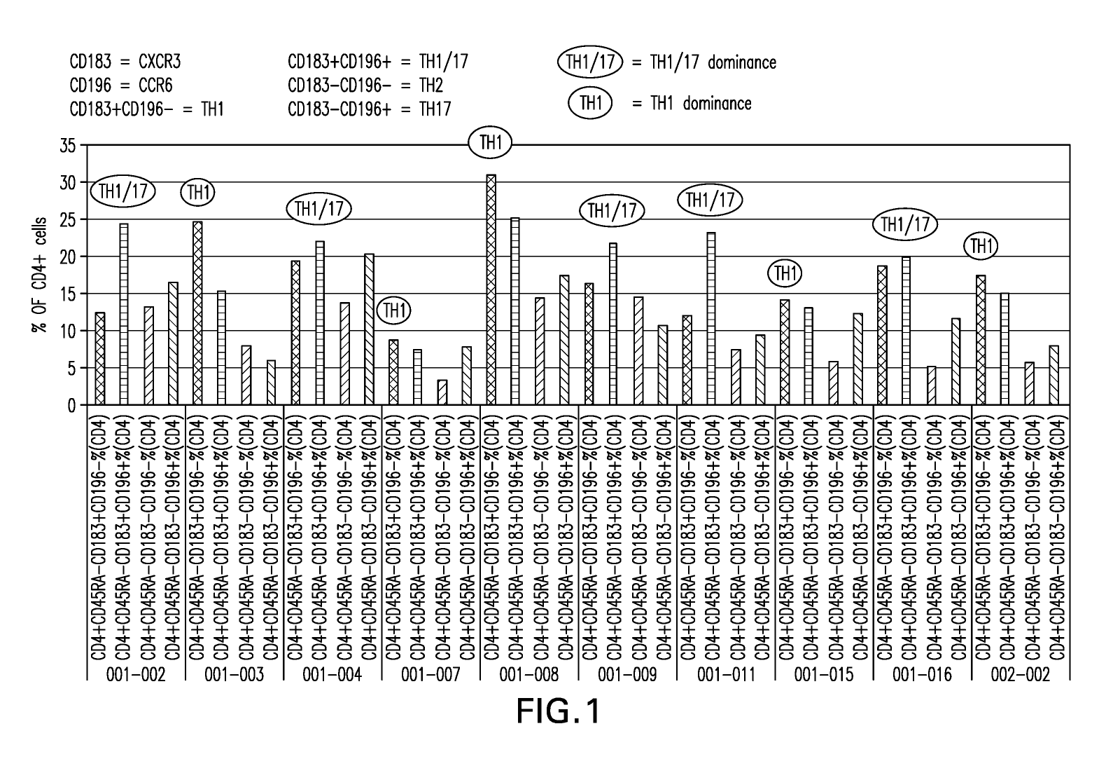

As shown in FIG. 1, flow cytometry data from 10 subjects enrolled in this study with a diagnosis of lower risk myelodysplastic syndrome indicated Th1 or Th1/17 phenotype dominance prior to trial treatment. The Th1/17 phenotype was dominant in 5 subjects; and the Th1 phenotype was dominant in 5 subjects.

The Th phenotypes were determined by surface markers of CD4+Th cells as detailed in the table below.

| Phenotype | CD183 (i.e. CXCR3) | CD196 (i.e. CCR6) |
|---|---|---|
| Th1 dominance | + | − |
| Th1/17 dominance | + | + |
| Th2 dominance | − | − |
| Th17 dominance | − | + |

### Example IV

### Tipifarnib Reduced Th1 Cytokine Production in Subjects with High Th1 Cytokine Levels

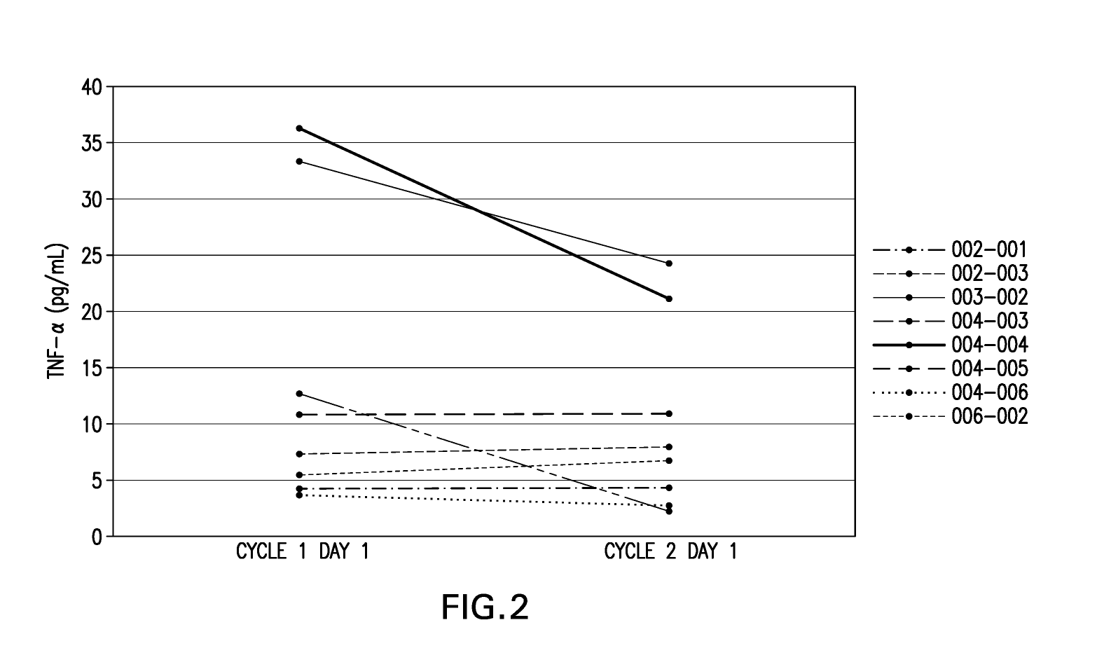

The serum cytokine levels were monitored in lymphoma subjects who received tipifarnib treatment at a starting dose of 900 mg on days 1-7 and 15-21 of 28 days treatment cycles. FIG. 2 shows measurements of TNF-alpha on Cycle 1 Day 1 and Cycle 2 Day 1 of tipifarnib for 8 subjects. As shown, tipifarnib induced downregulation of Th1 cytokine TNF-alpha production in subjects with high Th1 cytokine levels.

The instant application contains a Sequence Listing which has been submitted electronically in ASCII format and is hereby incorporated by reference in its entirety. Said ASCII copy, created on May 8, 2017, is named 649875-999010_SL.txt and is 7,080 bytes in size.

1. A method of treating myelodysplastic syndrome (MDS) in a subject, comprising administering a therapeutically effective amount of a farnesyltransferase inhibitor (FTI) to said subject, wherein said subject is characterized by Th1 dominance.

2. The method of claim 1, further comprising analyzing a sample from said subject to determine said subject is characterized by Th1 dominance prior to administering said FTI to said subject.

3. The method of claim 2, wherein said sample is
(i) a tumor biopsy or a body fluid sample;
(ii) a whole blood sample, a partially purified blood sample, a peripheral blood sample, a serum sample, a cell sample or a lymph node sample; or
(iii) peripheral blood mononuclear cells (PBMC).

4. (canceled)

5. (canceled)

6. The method of claim 2, comprising determining the expression level of a Th1 gene signature in said sample to be higher than a reference expression level of said Th1 gene signature; wherein the Th1 gene signature is selected from the group consisting of TBX21, STAT1, STAT6, CXCR3, CCR5, IFN-γ, TNF-α, IL-2, IL-12, and any combination thereof.

7. (canceled)

8. (canceled)

9. The method of claim 6, comprising
(i) determining the protein level of said Th1 gene signature using an immunehistochemistry (IHC) assay, an immunoblotting (IB) assay, an immunofluorescence (IF) assay, flow cytometry (FACS), or an Enzyme-Linked Immunosorbent Assay (ELISA); or
(ii) determining the mRNA level of said Th1 gene signature by Polymerase Chain Reaction (PCR), qPCR, qRT-PCR, RNA-seq, microarray analysis, SAGE, MassARRAY technique, next-generation sequencing, or FISH.

10. (canceled)

11. (canceled)

12. (canceled)

13. The method of claim 2, wherein said gene signature comprises TBX21 or CXCR3.

14. (canceled)

15. (canceled)

16. (canceled)

17. The method of claim 2, comprising determining the ratio of Th1 cells to Th2 cells in said sample to be higher than a reference ratio.

18. (canceled)

19. The method of claim 2, comprising determining that at least 20%, at least 30%, at least 40%, at least 50%, at least 60%, at least 70%, at least 80%, or at least 90% of the cells in said sample are Th1 cells.

20. (canceled)

21. (canceled)

22. The method of claim 2, comprising detecting a Th1 cytokine in said sample, said Th1 cytokine comprising IFN-γ, TNF-α, IL-2, or IL-12.

23. The method of claim 22, further comprising determining the level of said Th1 cytokine in said sample to be higher than a reference level.

24-33. (canceled)

34. The method of claim 1, wherein the FTI is selected from the group consisting of tipifarnib, arglabin, perrilyl alcohol, SCH-66336, L778123, L739749, FTI-277, L744832, CP-609,754, R208176, AZD3409, and BMS-214662.

35. The method of claim 24, wherein the FTI is tipifarnib.

36. (canceled)

37. The method of claim 1, wherein the FTI is administered at a dose of 1-1000 mg/kg body weight.

38. The method of claim 1, wherein the FTI is administered at a dose of 200-1200 mg twice a day.

39. The method of claim 38, wherein the FTI is administered at a dose of 600 mg twice a day.

40. The method of claim 38, wherein the FTI is administered at a dose of 900 mg twice a day.

41. (canceled)

42. The method of claim 1, wherein the FTI is administered daily for a period of one to seven days.

43. The method of claim 1, wherein the FTI is administered in alternate weeks.

44. The method of claim 1, wherein the FTI is administered on days 1-7 and 15-21 of a 28-day treatment cycle.

45. (canceled)

46. The method of claim 35, wherein tipifarnib is administered orally at a dose of 900 mg twice a day on days 1-7 and 15-21 of a 28-day treatment cycle.

47. (canceled)

48. The method of claim 1, further comprising administering a therapeutically effective amount of a second active agent or a support care therapy.

49. The method of claim 48, wherein said second active agent is a DNA-hypomethylating agent, a therapeutic antibody that specifically binds to a cancer antigen, a hematopoietic growth factor, cytokine, anti-cancer agent, antibiotic, cox-2 inhibitor, immunomodulatory agent, anti-thymocyte globulin, immunosuppressive agent, corticosteroid or a pharmacological derivative thereof.

50. The method of claim 1, wherein the MDS is lower risk MDS.

51. The method of claim 35, wherein tipifarnib is administered at a dose of 600 mg twice a day on days 1-7 and 15-21 of a 28-day treatment cycle.

52. The method of claim 35, wherein tipifarnib is administered at a dose of 300 mg twice a day for 3 of 4 weeks in repeated 4 week cycles.
